(19) United States
(12) Patent Application Publication
(10) Pub. No.: US 2025/0027956 A1
(43) Pub. Date: Jan. 23, 2025

(54) Improved Gamma-Secretase Inhibitor Screening Assays

(71) Applicants: VIB VZW, Gent (BE); Katholieke Universiteit Leuven, Leuven (BE)

(72) Inventors: Wim Annaert, Kontich (BE); Ragna Sannerud, Leuven (BE); Marine Bretou, Leuven (BE)

(21) Appl. No.: 18/711,255

(22) PCT Filed: Nov. 17, 2022
(86) PCT No.: PCT/EP2022/082340

§ 371 Date: May 17, 2024

(30) Foreign Application Priority Data
Nov. 18, 2021 (EP) 21208922.1

(51) Int. Cl. G01N 33/68; G01N 33/84; G01N 33/92

(52) U.S. Cl. CPC G01N 33/6896; G01N 33/84; G01N 33/92; G01N 2500/10; G01N 2800/2821

(57) ABSTRACT

Current application relates to the field of neurodegenerative diseases. Specifically, the present invention relates to screening methods to identify therapeutic candidates for the prevention and/or treatment of Alzheimer's disease. More particularly, said candidates overcome endolysosomal dysfunction resulting from an accumulation of APP-carboxyterminal fragments.

### CROSS REFERENCE TO RELATED APPLICATIONS

This application is a national phase entry under 35 U.S.C. § 371 of International Patent Application PCT/EP2022/082340, filed Nov. 17, 2022, designating the United States of America and published in English as International Patent Publication WO2023/089062 on May 25, 2023, which claims the benefit under Article 8 of the Patent Cooperation Treaty to European Patent Application Serial No. 21208922.1, filed Nov. 18, 2021, the entireties of which are hereby incorporated by reference.

### INCORPORATION BY REFERENCE

The ST.26 XML Sequence listing named “10488-10449-US_SequenceListingST26”, created on Nov. 16, 2022, and having a size of 20,480 bytes, is hereby incorporated herein by this reference in its entirety.

### TECHNICAL FIELD

Current application relates to the field of neurodegenerative diseases. Specifically, the present invention relates to screening methods to identify therapeutic candidates for the prevention and/or treatment of Alzheimer's disease. More particularly, said candidates overcome endolysosomal dysfunction resulting from an accumulation of APP-carboxyterminal fragments.

### BACKGROUND ART

Gamma (g)-secretases are intramembrane-cleaving proteases involved in various signalling pathways and diseases. G-secretases consist of 4 subunits. The catalytic activity of the complex is provided by presenilin (PSEN)1 or PSEN2, while three additional subunits, APH1 (A (long or short), B or C), nicastrin (NCST), and PEN-2 are needed to build a functional enzyme (De Strooper and Annaert 2010). Together with beta (b)-secretase, g-secretase complexes proteolyzes the amyloid precursor protein (APP), resulting in the production of a plethora of Amyloid beta (Ab) fragments with different lengths, e.g. Ab38, Ab40, Ab42, Ab43. Several studies point to long Ab peptides (Ab42 or longer) as key players in the oligomerization and initiation of aggregation of neurotoxic or amyloidogenic Ab species, which contribute directly to the development and progression of Alzheimer's disease (AD) (Haass and Selkoe 2007). Dominantly inherited mutations in the APP and PSEN genes, linked to early onset or familial Alzheimer's disease (AD), are shown to decrease g-secretase processivity (Szaruga et al 2017); shifting the production to longer, more aggregation prone Ab peptides and, therefore, accelerating Ab deposition in senile plaques in the brains of AD patients (Shioi et al 2007). G-secretase can process APP also in combination with alpha (a)-secretase. However, because a-secretase cleaves APP in the Ab domain and a-secretase cleavage thus precludes amyloid beta formation, the a-secretase pathway is considered to be part of the non-amyloidogenic pathway in APP processing.

Ever since the amyloid cascade hypothesis was proposed in 1991 (Hardy and Allsop 1991), much research on AD pathogenesis has focused on the mechanisms behind Ab generation and degradation. In line with this, several drug candidates have been developed, aiming at reducing Ab production especially amyloidogenic Ab fragments by inhibiting or modulating the key enzymes involved in the production of Ab. G-secretase inhibitors have been considered promising drug candidates against AD due to their ability to reduce g-secretase activity that directly leads to Ab production. However, there is a continuing debate to what extent toxic Ab is considered an initiating factor in the neurodegenerative cascade in AD, a driver in later stages, or both (De Strooper and Karran 2016; Selkoe and Hardy 2016). Moreover, clinical trials with g-secretase inhibitors have been halted due to lack of clinical efficacy and/or side effects.

Further basic research is thus needed to elucidate the underlying reasons and to provide new insights that will be fundamental in the development of improved therapies targeting g-secretase.

### SUMMARY OF INVENTION

The inventors of current application generated novel combined PSEN double knock-out (PSENdKO) and APP KO cells that allow them to systematically dissect the contribution of these genes in otherwise isogenic backgrounds. It is disclosed herein that a build-up of APP-carboxyterminal fragments (CTFs) caused by PSEN/g-secretase inactivation abruptly affects lysosomal calcium homeostasis, resulting in a cascade of events affecting endosomal recycling and sorting compartments and, consequently, prompting a collapse of the normal endolysosomal maturation. A knock-out of APP rescues these chronological events whereas they are mimicked by re-introducing a membrane-tethered APP cytoplasmic domain, narrowing down the effect to downstream APP-CTF signalling. Mechanistically, this can be explained by the accumulation of APP-CTFs at or near late endosome/lysosome (LE/Lys)-endoplasmic reticulum (ER) contact sites, affecting lysosomal calcium re-filling from the ER. The data herein disclosed imply for the first time the requirement for balanced APP-CTF levels in the functionality of LE/Lys-ER membrane contact sites (MCSs) to maintain endolysosomal homeostasis. These toxic effects make APP-CTF an important therapeutic target to combat AD progression at a very early stage, long before the appearance of amyloid plaques.

Therefore, in a first aspect current document discloses a method to select an improved APP processing modulator from a collection of APP processing modulators, comprising the steps of:
a) administering an APP processing modulator from said collection to one or more cells expressing a gamma-secretase complex and comprising a (poly)peptide SEQ ID No. 1 or a homologue thereof with at least 95% amino acid identity or a fragment thereof;
b) quantifying the level of APP-carboxyterminal fragment (CTF) in the one or more cells before and after administering the gamma-secretase inhibitor;
c) optionally determining the Ca2+ level and/or the cholesterol level in the lysosomes of the one or more cells before and after administering the gamma-secretase inhibitor;
   identifying said APP processing modulator as an improved APP processing modulator if the APP-CTF level is at most 50% increased after the administration of the g-secretase inhibitor compared to before said administration and optionally if the Ca2+ level in the lysosomes is at most 20% decreased and/or the cholesterol level in the lysosomes is at most 25% increased after the administration of the g-secretase inhibitor compared to before said administration.

In a second aspect, a screening method is provided for identifying an improved APP processing modulator, comprising the steps of:
a) providing one or more cells expressing a functional gamma-secretase complex and comprising a (poly)peptide SEQ ID No. 1 or a homologue thereof with at least 95% amino acid or a fragment thereof;
b) quantifying Ab peptides with a length of 38, 40, 42 and/or 43 amino acids produced in said one or more cells before and after administering a test compound;
c) quantifying before and after administering the test compound the level of APP-CTF in the one or more cells and/or the Ca2+ level in the lysosomes of the one or more cells and/or the cholesterol level in the lysosomes of the one or more cells;
   identifying said test compound as an improved APP processing modulator if after the administration in step b) reveals a statistically significant increase in the ratio of Ab38/Ab42, Ab40/Ab42, Ab40/Ab43 or of Ab(38+40)/Ab(42+43) compared to the situation before administering said test compound and if the administration in step c) reveals an at most 50% increased APP-CTF level and/or an at most 20% decrease in lysosomal Ca2+ level and/or an at most 25% increase lysosomal cholesterol level after the administration of the test compound compared to before said administration.

Several embodiments of the above methods are disclosed herein. In one embodiment, the APP-CTF level is quantified via immune-based assays. In another embodiment, the APP-CTF is fluorescently labelled and the APP-CTF level corresponds to the APP-CTF fluorescent signal. In another embodiment, the Ab peptides are quantified via immune-based assays. In yet another embodiment, the Ca2+ and/or cholesterol level in the lysosomes is determined by an image-based assay. In a particular embodiment, the lysosomes are visualised by LAMP1. In another particular embodiment, the one or more cells in the above disclosed methods are mammalian cells. In yet another embodiment, the improved APP processing modulator from the methods disclosed herein is a therapeutic candidate for the prevention and/or treatment of Alzheimer's disease.

In a third aspect, current disclosure provides a stimulator or enhancer of cholesterol efflux from the late endosome and/or lysosome compartments in a cell for use to treat Alzheimer's disease.

### TECHNICAL PROBLEM

Drug discovery efforts have been focussing on inhibiting g-secretase activity in the brain to overcome the production of amyloidogenic Ab fragments. However, it is more and more questioned whether Ab deposition is the initiation factor in dementias such as Alzheimer's disease, while clinical trials revealed that g-secretase inhibitors are not effective.

### SOLUTION TO PROBLEM

The inventors of current application dissected the cell biological events at play during chronic inhibition of g-secretase activity in a chronological way. It is herein disclosed that the first event is an accumulation of APP-CTFs which on its turn affects lysosomal calcium and cholesterol homeostasis and leads to endolysosomal demise and neuronal death.

### ADVANTAGEOUS EFFECTS OF INVENTION

Based on the novel insights herein described it is now clear that a g-secretase activity intervention and APP processing at large must avoid the accumulation of APP-CTFs at all times. Hence, newly designed or discovered APP processing modulators should be additionally screened on their effect on APP-CTF accumulation and/or lysosomal Ca2+ and/or cholesterol levels.

### BRIEF DESCRIPTION OF DRAWINGS

FIGS. 1A-1J Chronic g-secretase inhibition identifies a unique chronology in endolysosomal defects

FIG. 1A is an immunoblot of APP full length (FL) and APP-C-terminal fragments (CTFs) detected in WT MEFs treated with DMSO or DAPT (1 μM for 1-4 days).

FIG. 1B shows the Mander's colocalization coefficient for APP-Lamp1.

FIG. 1C shows the lysosomal Ca2+ content of Fura-2AM loaded cells treated with DMSO or DAPT (1 μM for 1-4 days) and challenged with 100 μM GPN (340/380). Lysosomal Ca2+ levels are reduced in DAPT treated cells, starting from day 1 (Data pooled from N=3 independent experiments, mean±SEM). Data were normalized to their respective DMSO controls (indicated by the line at 1, with n=38, 38, 39 and 51 for the corresponding controls). Multiple Anova Kruskal-Wallis with Dunn's post-test was applied.

FIG. 1D shows the Mander's colocalization coefficient for Filipin-Lamp1. APP-CTF accumulation in Lamp1-compartments is detected at day 1 onward (FIG. 1B), while this occurs for cholesterol (filipin staining) from day 2 onwards (FIG. 1D) (pooled data from N=2 experiments, with, from left to right, n=112, 109, 120, 111, 127, 123, 120 and 127 cells).

FIG. 1E shows that VPS35 compartments significantly enlarge from day 3 onwards (pooled data from N≥2 experiments, with n=60, 63, 70, 62, 68, 65, 84 and 83 cells, and normalized to DMSO).

FIG. 1F shows that EEA1 compartments enlarge from day 4 (pooled data from N=2 experiments, with n=113, 114 96, 103, 72, 93, 77 and 66 cells, and normalized to DMSO).

FIGS. 1G-1J show the Mander's colocalization coefficient for VPS35-Lamp1 (FIG. 1G), EEA1-Lamp1 (FIG. 1H), APP-VPS35 (FIG. 1I) and VPS35-EEA1 (FIG. 1J). Endolysosomal collapse is detected from day 3 onward (pooled data from N=2 experiments, with n=97, 114, 96, 103, 73, 93, 77 and 66 cells, normalized to DMSO).

Multiple Anova Kruskal-Wallis with Dunn's post-test was applied.

FIGS. 2A-2D APP depletion normalizes endolysosomal defects

FIGS. 2A-2C shows the lysosomal Ca2+ content of Fura-2AM loaded cells challenged with 100 μM GPN (340/380) (mean±SEM). Lysosomal Ca2+ levels are reduced in PSENdKO clones and restored after re-expression of hPSEN1 WT (A) (Pooled data pooled from N=3 experiments, normalized to PSENdKO). Knocking out APP restores lysosomal Ca2+content of PSENdKO cells (FIG. 2B) (pooled data from N>2 experiments). Lysosomal Ca2+ content is reduced in WT cells treated with DAPT or InhibitorX (4 days), but not in APPKO cells (C FIG. 2) (pooled data from N=3 experiments and normalized to DMSO-treated WT or APPKO cells).

FIG. 2D is an immunoblot of WT or WT-APPKO cells treated with either DAPT (1μM) or inhibitor X (1 μM). APPNL-G-F neurons treated with DAPT were used as a reference for the different APP-CTFs.

FIGS. 3A-3K

FIGS. 3A-3C shows that endolysosomal collapse, indicated by increased colocalization of EEA1 with Lamp1 (FIG. 3A), VPS35 with EEA1 (FIG. 3B) and VPS35 with Lamp1 (FIG. 3C) in PSENdKO cells is rescued upon hPSEN1 expression or APP depletion. (Mander's colocalization coefficient; pooled data from N≥2 experiments with n=73, 152, 66 and 64 cells (FIG. 3A), n=73, 152, 66 and 64 cells (FIG. 3B) and n=150, 234, 158 and 64 cells (FIG. 3C)). Data were normalized to PSENdKO (normalized to 1, or median of data obtained per experiment).

FIGS. 3D-3F shows that re-expression of hPSEN1 or APP depletion in PSENdKO background normalizes the size of EEA1- (FIG. 3D) and VPS35- (FIG. 3F) positive endosomes. Lamp1 total area is normalized to WT levels upon hPSEN1 expression, but not APP depletion (FIG. 3E) (pooled data from N≥2 experiments with n=73, 161, 66 and 75 cells (FIG. 3D), n=150, 243, 160 and 75 cells (FIG. 3E) and n=138, 200, 145 and 75 cells (FIG. 3F)).

FIGS. 3G and 3J show (based on immunostaining) that APP accumulates in VPS35- (FIG. 3G) and in Lamp1- (FIG. 3J) positive organelles in PSENdKO, as seen by the increased Mander colocalization coefficient. Expression of hPSEN1 WT abolished APP accumulation back to WT levels (Data pooled from N=4 independent experiments, with, from left to right, n=77, 83 and 92 cells). When indicated, multiple Anova Kruskal-Wallis with Dunn's post-test was applied.

FIG. 3H shows that the number of MVBs per cell was not significantly altered, while Figure K shows that the percentage of electron lucent MVBs is increased in PSENdKO cells as compared to the different rescued cell lines (mean±SEM). Mann-Whitney test was applied.

FIG. 3I shows the percentage of cells exhibiting repolarized Cav1 in the different conditions (Data pooled from N=5, 7, 5 and 2 independent experiments, mean±SEM). Multiple Anova Kruskal-Wallis with Dunn's post-test was applied.

FIGS. 4A-4F Expression of APP-CTFs is sufficient to induce endolysosomal defects

FIGS. 4A-4B show that re-introducing a- or b-CTFs in PSENdKO-APPKO cells equally results in the accumulation of enlarged EEA1 (FIG. 4A) and VPS35 (FIG. 4B) positive endosomes (pooled data from N=2 experiments, normalized to PSENdKO cells, with n=45, 49 and 50 cells from left to right).

FIGS. 4C-4D show the colocalization of VPS35 with EEA1 (FIG. 4C) or Lamp1 (FIG. 4D) increases upon expression of either CTFs in PSENdKO-APPKO cells (pooled data from N=2 experiments, with n=47, 49 and 50 cells).

FIG. 4E shows the percentage of cells exhibiting repolarized Cav1 in PSENdKO-APPKO cells expressing no CTFs, a-(C83) or b-(C99) CTFs or APP695. (Data pooled from N=2-3 independent experiments, mean±SEM). Multiple Anova Kruskal-Wallis with Dunn's post-test was applied.

FIG. 4F shows the quantification of lysosomal Ca2+ response of Fura-2AM loaded cells challenged with 100 μM GPN (340/380). Expression of either CTFs in PSENdKO-APPKO cells recapitulates the lysosomal Ca2+ defect observed in PSENdKO cells (pooled data from N=2 experiments, with n=27, 36, 32, 23 and 25, from left to right; mean±SEM, normalized to PSENdKO). Multiple Anova Kruskal-Wallis with Dunn's post-test was applied.

FIGS. 5A-5M Membrane anchoring of the APP intracellular domain is sufficient to induce endolysosomal defects

FIG. 5A shows the amino acid sequence of control (mCtrl) and myristyolated AICD (mAICD) constructs, with mutations indicated.

FIGS. 5B-5D show that re-expression of mAICD is sufficient to cause enlargement of EEA1- (FIG. 5B), Lamp1- (FIG. 5C) and VPS35- (FIG. 5D) positive compartments (pooled data from N=3 experiments with n=59, 65 and 70 cells from left to right for (FIG. 5B) and n=46, 57 and 52 cells for (FIGS. 5C-5D); data normalized to mCtrl).

FIGS. 5E-5F show Mander's colocalization coefficient. Increased colocalization of VPS35 with EEA1 (FIG. 5E) or with Lamp1 (FIG. 5F) suggests a delay in endolysosomal maturation in mAICD expressing cells (Data pooled from N≥3 experiments with n=59, 65 and 70 cells (E), n=106, 122 and 125 cells (FIG. 5F)).

FIG. 5G shows that lysosomal Ca2+ response is reduced in cells expressing mAICD, and normalized back to Ctrl levels for mutant mAICD (pooled data from N=2 experiments and normalized to mCtrl; mean±SEM). Multiple Anova Kruskal-Wallis with Dunn's post-test was applied for FIGS. 5B-5F; for FIG. 5G Mann-Whitney test was applied.

FIG. 5H shows the percentage of cells exhibiting repolarized Cav1 in the different conditions (Data pooled from N=2 independent experiments, mean±SEM, Mann Whitney test was applied).

FIG. 5I shows the lysosomal Ca2+ content of WT Fura-2AM loaded cells treated with DMSO or YM201636 (2 μM, 2 h) and challenged with 100 μM GPN (340/380). Lysosomal Ca2+ levels are reduced in YM201636-treated cells (Data pooled from N=3 independent experiments, mean±SEM, Mann Whitney test was applied).

FIG. 5J illustrates that imatinib treatment partially corrects the altered cargo sorting detected in PSENdKO cells, as evidenced by the decreased colocalization of VPS35 with Lamp1 (n=26 and 21 cells for PSENdKO cells treated with DMSO or Imatinib, Mann Whitney test was applied).

FIG. 5K shows that the imatinib treatment relieved the lysosomal Ca2+ defect, normally observed in PSENdKO cells, in a dose dependent manner (mean±SEM; the upper panel depicts the mean Lysosomal Ca2+ responses in function of the imatinib concentration used). Multiple Anova Kruskal-Wallis with Dunn's post-test was applied.

FIGS. 5L-5M show that the APP accumulation in Lamp1-compartment was not relieved upon Imatinib treatment (FIG. 5L), contrary to its accumulation in VPS35-compartments (FIG. 5M), as evidenced by the Manders colocalization coefficients.

FIGS. 6A-6L Reduced lysosomal Ca2+ results from altered LE/Lys-ER membrane contacts

FIG. 6A shows the Ca2+ response (area under curve) detected in Gcamp6 TRPML1 KI/KI PSEN1 WT or KO neurons (div 7) loaded with Fura-2AM and challenged with MLSA1 (20 μM) and GPN (500 μM) (mean±SEM, Mann Whitney test was applied).

FIG. 6B shows that lysosomal Ca2+ refilling is impaired in PSENdKO cells and is normalized upon expression of either hPSEN1 or APP depletion (pooled data from N=3, 5, 3 and 2 experiments).

FIGS. 6C-6E show the line scans of an Airyscan imaging of Lamp1 and APP in Sec61-GFP transfected cells. Depicted are contact sites visualized using Airyscan in PSENdKO (FIG. 6C), PSENdKO-APPKO cells expressing C99 (FIG. 6D) or mAICD (FIG. 6E).

FIGS. 6F-6G show the line scans of an immunostaining of APP-in VAPB- and Stard3 transfected cells. Depicted are contact sites visualized using Airyscan in PSENdKO (FIG. 6F) and PSENdKO-APPKO expressing C99 cells (FIG. 6G).

FIGS. 6H-6I show the line scans of an Airyscan imaging of PSENdKO-APPKO cells transfected with Sec61-mcherry and APP-YFP (FIG. 6H) or C99-GFP (FIG. 6I) and upon hypotonic treatment.

FIG. 6J shows the line scans of an Airyscan imaging of Lamp1, APP and HSP60 in PSENdKO-APPKO expressing C99 cells.

FIGS. 6K-6L show the line scans of an Airyscan imaging of PSENdKO-APPKO cells transfected with APP-YFP (FIG. 6K) or C99-YFP (FIG. 6L) and treated with Mitotracker and upon hypotonic treatment. Enlarged organelles containing APP (FIG. 6K) or C99 (FIG. 6L) were not found near mitochondria, as visualized on the line scans. Multiple Anova Kruskal-Wallis with Dunn's post-test was applied.

FIGS. 7A-7I. Expression of membrane-tethered APP fragments affect LE/Lys-ER MCS morphology and LE/Lys dynamics

FIGS. 7A-7E show the quantification of MCS lengths (mean±SEM) of KDEL-HRP transfected cells analysed with TEM. Only PSENdKO cells (FIG. 7A) and PSENdKO-APPKO cells expressing mAICD (FIG. 7D) displayed extended LE/Lys-ER contacts. MCS lengths are normalized upon expression of hPSEN1 or APP depletion in a PSENdKO background, or when expressing mutant mAICD in a PSENdKO-APPKO background.

FIGS. 7F-7G illustrate the dynamics of Lysotracker positive LE/Lys in cells transfected with Sec61-GFP. FIG. 7F shows a comparison of each motility frequency in PSENdKO, PSENdKO+hPSEN1, PSENdKO+APPKO, PSENdKO+APPKO+mAICD and PSENdKO+APPKO+mAICDY682A-Y687A (pooled data from n=13, 10, 9, 4 and 9 cells, from left to right). FIG. 7G is a plot of the MSD over time showing a decreased organelle motility in cells accumulating (PSENdKO) or expressing mAICD (PSENdKO-APPKO+mAICD).

FIG. 7H shows that the reduced organelle motility results in decreased average diffusion coefficients.

FIG. 7I shows the percentage overlap between lysosomes and ER. Indicated are the number of planes and corresponding cell numbers.

The plots represent mean±SEM. Mann-Whitney test was applied. For FIGS. 7B-7E and FIG. 7I, one-way Anova and Multiple Anova Kruskal-Wallis with Dunn's post-test were applied, respectively.

FIGS. 8A-8L Cholesterol egress corrects lysosomal Ca2+ defects

FIGS. 8A-8C show the Mander's colocalization coefficient of Filipin-Lamp1 (FIG. 8A), Filipin-APP (FIG. 8B) and APP-Lamp1 (FIG. 8C) (pooled data from N=2 experiments, with n=71, 80, 61, 65, 73, 46, 78 and 67 cells, Multiple Anova Kruskal-Wallis with Dunn's post-test was applied).

FIG. 8D shows that NPC1 expression in PSENdKO cells reduces APP accumulation in Lamp1-positive compartments (Manders colocalization coefficient, data pooled from N=2 experiments).

FIG. 8E confirms a reduced cholesterol level in Lamp1-positive compartments upon NPC1 ovexpression in PSENdKO cells (Manders colocalization coefficient, data pooled from N=2 experiments).

FIGS. 8F-G show that NPC1 expression in PSENdKO cells restores endolysosomal maturation and recycling (Manders colocalization coefficient, data pooled from N=3 experiments).US

FIGS. 8H-J show that HPCγD treatment of PSENdKO cells reduces cholesterol accumulation (FIG. 8I), and APP-CTFs fragments (FIG. 8H) in Lamp1-positive compartments and restores VPS35-mediated recycling (FIG. 8J) (Manders colocalization coefficient, data pooled from N=2 to 3 experiments).

FIG. 8K shows that HPCyD treatment reduces the size of EEA1 compartments in PSENdKO cells (data pooled from N=2 experiments).

FIG. 8L shows that overexpressing NPC1-Flag in PSENdKO cells or treating PSENdKO cells with 2-hydroxypropyl-γ-cyclodextrin (HPγCD, 1 mM, 4days) alleviates lysosomal Ca2+ defects (pooled data from N≥2 experiments, mean±SEM).

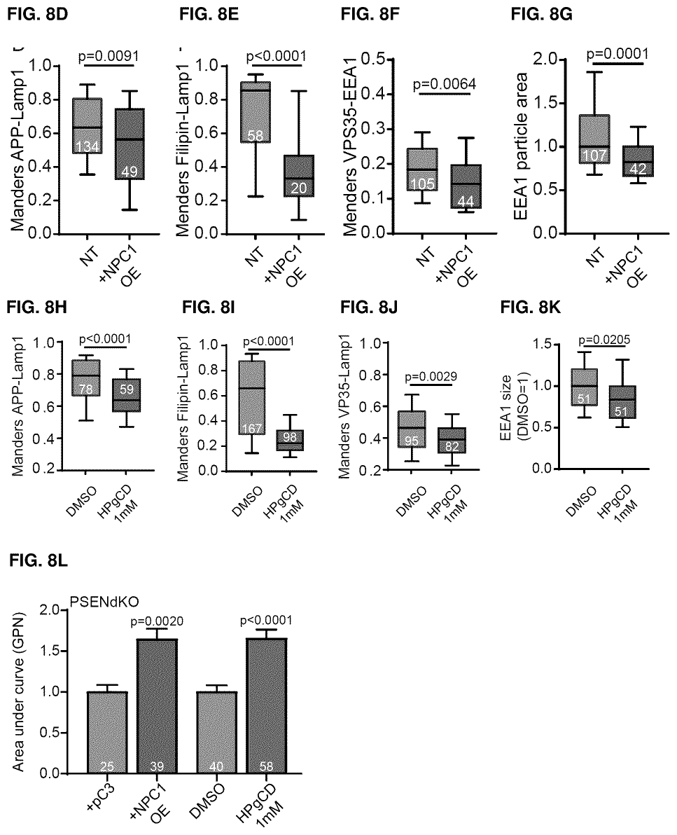

Mann-Whitney test was applied in FIG. 8D-8L.

### DESCRIPTION OF EMBODIMENTS

As used herein, each of the following terms has the meaning associated with it in this section. The articles “a” and “an” are used herein to refer to one or to more than one (i.e. to at least one) of the grammatical object of the article. By way of example, “an element” means one element or more than one element. “About” as used herein when referring to a measurable value such as an amount, a temporal duration, and the like, is meant to encompass variations of ±20% or ±10%, more particularly ±5%, even more particularly ±1%, and still more particularly ±0.1% from the specified value, as such variations are appropriate to perform the disclosed methods. The term “abnormal” when used in the context of organisms, tissues, cells or components thereof, refers to those organisms, tissues, cells or components thereof that differ in at least one observable or detectable characteristic (e.g. age, treatment, time of day, etc.) from those organisms, tissues, cells or components thereof that display the “normal” (expected) respective characteristic. Characteristics which are normal or expected for one cell or tissue type, might be abnormal for a different cell or tissue type. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. Unless specifically defined herein, all terms used herein have the same meaning as they would to one skilled in the art of the present invention. Practitioners are particularly directed to Sambrook et al. 2012 Molecular Cloning: A Laboratory Manual, 4th ed., Cold Spring Harbor Press, Plainsview, New York; and Ausubel et al. 2012 Current Protocols in Molecular Biology (Supplement 100), John Wiley & Sons, New York, for definitions and terms of the art. The definitions provided herein should not be construed to have a scope less than understood by a person of ordinary skill in the art.

In this document, the pathological contribution of excess APP-CTFs on causing endolysosomal collapse in neurodegenerative disorders is unveiled and additionally the chronology in said process is reported. The inventors identified a decreased Ca2+ content in the late endosome/lysosome (LE/Lys) as primary event in the cascade leading to APP-CTF mediated neurotoxicity. Once Ca2+ homeostasis is dysregulated, cholesterol starts to build-up in LE/Lys, which, in turn, affects endosomal recycling and finally results in enlarged early endosomal compartments. The congruent increase in the co-localization of lysosomal and endosomal markers underscores a progressive collapse of the endolysosomal compartment.

Endosomes and lysosomes are membrane-bound organelles crucial for the normal functioning of the eukaryotic cell. The primary function of endosomes relates to the transportation of extracellular material into the intracellular domain. In addition, endosomes also play an important role in cell signalling and autophagy. Lysosomes, on the other hand, act as the final compartment in the endocytosis (as well as autophagy) pathway, and play a critical role in the degradation and proteolysis of internalized macromolecules, dysfunctional proteins and organelles.

Upon internalisation, cargo is delivered to an early endosome (EE). The EE acts as the primary sorting station of the endocytic pathway. A subset of molecules is recycled back to the plasma membrane via either a direct pathway or via recycling endosomes. The remaining molecules are transported further into the cell. The EE matures into a late endosome (LE) as it moves towards the perinuclear area along microtubules. The nascent LE grows in size by undergoing homotypic fusion reactions and acquiring additional cargo from intraluminal vesicles (ILVs). LEs serve as a secondary sorting station, from which cargo can be delivered to various destinations. A substantial fraction of the internalized molecules will be transported to lysosomes (Lys) for degradation. The LE fuses with a classical dense lysosome to form a transient hybrid organelle, known as an endolysosome, in which active degradation of endocytosed cargo occurs. Endolysosomes further maturate to form classical dense lysosomes, which act as storage organelles for membrane components and hydrolases.

The late endosome/lysosome (LE/Lys) as used herein refers to a set of intracellular membranous compartments that dynamically interconvert, and which is comprised of late endosomes and lysosomes.

“APP-CTF” as uses herein refers to membrane bound APP fragments, more particularly to APP C99 as disclosed in SEQ ID No. 2 and/or to APP-C83 as disclosed in SEQ ID No. 3.

“APP C99” is also referred to herein as beta-cleaved APP-CTF or APP-beta-CTF or APP-b-CTF.

“APP C83” is also referred to herein as alpha-cleaved APP-CTF or APP-alpha-CTF or APP-a-CTF.

“APP” stands for Amyloid precursor protein and is referred to herein as the protein as disclosed in SEQ ID No. 1.

It is herein demonstrated that APP-CTFs function on the membrane contact sites (MCS) between the LE/Lys and the endoplasmic reticulum (ER). So in contrast to the rationale behind the multiple attempts to pharmacologically block g-secretase activity, the inventors found that a timely and full processing of APP is required to safeguard the critical balance in LE/Lys-ER communication. Indeed, inhibiting g-secretase activity feeds a prolonged signalling cascade that originates from APP's cytosolic domain and distorts inter-organellar communication and ultimately leads to endolysosomal collapse.

The findings herein reported elucidate the puzzle of APP-mediated signalling and challenge previous findings related to APP-CTF function and localization. First, it is demonstrated that not initiation, but abrogation of downstream signalling of APP is required to maintain endolysosomal homeostasis. This is opposite to other substrates such as Notch, N-cadherin and ErbB4, where g-secretase cleavage constitutes an intrinsic part of key signalling mechanisms (Bray 2016; Jurisch-Yaksi et al 2013; Kopan and Ilagan 2004; Marambaud et al 2003; Sardi et al 2006).

Secondly, and as opposed to recent studies (Hung and Livesey 2018; Kim et al 2016; Kwart et al 2019; Xu et al 2016), endolysosomal defects were induced by excess of either alpha- and beta-cleaved APP-CTFs. The beta-centred view might have originated from the use of neuronal cultures known for their predominant amyloidogenic processing, masking the more subtle increased APP-alpha-CTF levels (Hung and Livesey 2018; Kwart et al 2019), or overexpression strategies (Xu et al 2016). Alternatively, as alpha-shedding mainly occurs at the cell surface, the resulting APP-alpha-CTFs may not reach LE/Lys. Most importantly, given that membrane tethering of the AICD fragment through myristyolation is sufficient to recapitulate the spectrum of endolysosome defects in combined PSEN and APP deficient cells, there is no further ground for attributing differential effects to alpha- and beta-APP-CTFs. As such, our data question the existence of a direct nuclear signalling role for AICD, as proposed previously (Alves da Costa et al 2006; Cao and Sudhof 2001; Kimberly et al 2001; Muller et al 2007; Pardossi-Piquard et al 2005).

Based hereon, the invention relates to methods for selecting or identifying a therapeutic candidate for the prevention and/or treatment of neurodegenerative diseases, more particularly of neurodegenerative diseases wherein the lysosomal function is compromised, most particularly Alzheimer's disease or Niemann-Pick Disease Type C, even most particularly Alzheimer's disease.

In a first aspect, the therapeutic candidate is selected from a collection of compounds that modulate the processing of APP, more particularly modulate the alpha-, beta- and/or gamma-secretase activity, even more particularly the gamma-secretase activity. Said collection can be an assortment of compounds previously reported (e.g. in the art) as APP processing modulators or can be a selection of a chemical library pre-screened for APP processing modulation.

“Processing of APP” as used herein refers to the enzymatic degradation or truncation of APP. “Modulating the processing of APP” refers to a statistically significantly different processing of APP in the presence of a compound compared to the situation when the compound is absent.

“Modulating alpha-, beta-, and/or gamma-secretase activity” refers to a statistically significantly different activity of the secretase enzymes in the presence of a compound compared to the situation when the compound is absent. In a particular embodiment, modulating alpha-, beta-, and/or gamma-secretase activity means statistically significantly inhibiting or reducing alpha-, beta-, and/or gamma-secretase activity.

The terms “gamma-secretase”, “gamma-secretase protein complex” and “gamma-secretase complex” refer to a protein complex used herein comprising at least four protein molecules, where at least one of the protein molecules provides a catalytic site for cleavage of a polypeptide substrate having a gamma-secretase cleavage sequence, and wherein the protein molecules are PSEN1 or PSEN2, Aph1a or Aph1b, NCT, and/or PEN2. The protein molecules that comprise the gamma (g)-secretase protein complex may associate with each other. Additionally, the g-secretase protein complex may also include non-proteinaceous molecules, such as vitamins, ATP, or divalent cations. In one embodiment, the g-secretase as used herein is a functional or wild-type g-secretase, meaning that the g-secretase processes APP fragments in a wild-type manner.

Alpha (a)-secretases are a family of proteolytic enzymes that cleave APP in its transmembrane region, i.e. the fragment that gives rise to the Alzheimer's disease-associated peptide amyloid beta (Ab) when APP is instead processed by b-secretase and g-secretase. Upon cleavage by a-secretases, APP releases its extracellular domain—a fragment known as soluble(s) APPalpha—into the extracellular environment in a process known as ectodomain shedding. The remaining membrane bound APP-C83 fragment is then further cleaved by g-secretase into the P3 peptide and the APP intracellular domain (AICD). Alpha secretase activity resides among members of the ADAM (‘a disintegrin and metalloprotease domain’) family, which are expressed on the surfaces of cells and anchored in the cell membrane. Non-limiting examples of a-secretases are ADAM10, ADAM17, ADAM9, ADAM19.

Beta (b)-secretase also known as beta-site APP cleaving enzyme or BACE1 initiates the amyloidogenic pathway. Extracellular cleavage of APP by BACE1 creates a soluble extracellular fragment and a cell membrane-bound fragment referred to as APP-C99. Cleavage of APP-C99 within its transmembrane domain by g-secretase releases the intracellular domain of APP (AICD) and produces Ab. Since g-secretase cleaves APP closer to the cell membrane than BACE1 does, it removes a fragment of the Ab peptide.

A non-limiting example as read-out for APP processing or alpha (a)-, beta (b)- and/or gamma (g)-secretase activity, is the detection and quantification of APP fragments, more particularly for the different Ab fragments. Antibodies and accompanying immune-based assays to detect and quantify said fragments are available in the art (see also further below).

In one embodiment, said compounds that modulate are inhibitors, meaning that said compounds statistically significantly reduce the processing of APP or the alpha-, beta-, and/or gamma-secretase activity.

In another embodiment, said compounds that modulate are stabilizers, meaning that upon administration of said compound the enzyme/substrate complex has an increased stability as compared to the same test conditions without administered compound. In a particular embodiment, said compounds that modulate are g-secretase stabilizers or g-secretase stabilizing compounds and refer to compounds which, upon administration to a system (e.g. cell-based system comprising one or more cells) comprising a g-secretase and an APP/Ab substrate, provide an increased stability of the enzyme/substrate complex, as compared to the same test conditions without administered compound. With “increased stability”, it is meant that the enzyme/substrate complex has a longer half-life, higher melting temperature (Tm), improved binding properties, and/or more efficient processing of Ab cleavage. “Increased” stability refers to a statistically significant change compared to the control in the absence of the compound, particularly, but not by way of limitation, at least of about 5%, at least of about 10%, at least of about 15%, at least of about 20%, at least of about 25%, at least of about 30%, at least of about 35%, at least 10 of about 40%, at least of about 45%, at least of about 50%, at least of about 60%, at least of about 70%, at least of about 80%, or at least of about >90%. More specifically, the higher the enzyme/substrate complex its stability, the better its processivity to cleave substrate, hence, the higher the resulting amount of shorter Ab peptides. G-secretase stabilizers as used herein thus induce a statistically significant shift in the production of Ab fragments, more particularly a statistically significant increase in the ratio of Ab38/Ab42, Ab40/Ab42, Ab40/Ab43 and/or of Ab (38+40)/Ab (42+43) compared to the situation before administering said g-secretase stabilizer. When an increased ratio is obtained, the resulting amount of shorter Ab peptides will be higher than the resulting amount of “less-processed” or longer Ab peptides, which indicates that the g-secretase substrate complex was more active, and therefore showing increased stability.

Lower stability of the g-secretase complex means that there is a lower processivity of the complex and a faster release of Ab peptides. Hence, a lower stability leads to the production of longer and more amyloidogenic Ab peptides as compared to the wild type g-secretase complex. Pathogenic PSEN or APP mutations destabilize g-secretase/Ab complexes and thereby promote generation of longer Ab peptides (Szaruga et al Cell 2017). Similarly, destabilization of wild type g-secretase/Ab complexes by temperature, compounds, or detergent promotes release of amyloidogenic Ab. In addition, several FAD-causing APP mutations, known to affect the g-secretase processivity of APP, destabilize the g-secretase/Ab interaction and prime “de novo long Ab substrates” for dissociation.

In this document it is described that APP processing modulators affecting the Ab ratio in a positive way, meaning less amyloidogenic long fragments and more short non-amyloidogenic Ab fragments, could still lead to an accumulation of APP-CTFs and hence neurotoxic effects. Current disclosure provides solutions by further analysing the effects of the APP processing modulators on APP-CTF levels and/or on events downstream of APP-CTF accumulation.

Therefore a method is disclosed of selecting an improved APP processing modulator from a collection of compounds having known APP processing modulating activity, comprising the steps of:
a) administering an APP processing modulator from said collection to one or more cells;
b) quantifying the level of APP-carboxyterminal fragments (CTFs) in the one or more cells before and after administering the APP processing modulator;
c) optionally determining the Ca2+ level and/or the cholesterol level in the lysosomes of the one or more cells before and after administering the APP processing modulator;
   identifying said APP processing modulator as an improved APP processing modulator if the APP-CTF level is at most 50%, at most 40%, at most 30%, at most 25%, at most 20%, at most 15%, at most 10% or at most 5% or not statistically significantly increased after the administration of the APP processing modulator compared to before said administration and optionally if the Ca2+ level in the lysosomes is at most 20%, at most 15%, at most 10% or at most 5% or not statistically significantly decreased and/or the cholesterol level in the lysosomes is at most 25%, at most 20%, at most 15%, at most 10% or at most 5% or not statistically significantly increased after the administration of the APP processing modulator compared to before said administration. In one embodiment, said one or more cells express a gamma-secretase complex and comprising a (poly)peptide SEQ ID NO:1 or a homologue with 95% amino acid identity over the full length thereof, or any fragment thereof.

Also disclosed is a method of selecting an improved APP processing modulator from a collection of compounds with known APP processing modulating activity, comprising the steps of:
a) administering an APP processing modulator from said collection to one or more cells;
b) determining the Ca2+ level and/or the cholesterol level in the lysosomes of the one or more cells before and after administering the APP processing modulator;
   identifying said APP processing modulator as an improved APP processing modulator if the Ca2+ level in the lysosomes is at most 20%, at most 15%, at most 10% or at most 5% or not statistically significantly decreased and/or the cholesterol level in the lysosomes is at most 25%, at most 20%, at most 15%, at most 10% or at most 5% or not statistically significantly increased after the administration of the APP processing modulator compared to before said administration. In one embodiment, said one or more cells express a gamma-secretase complex and comprising a (poly)peptide SEQ ID NO:1 or a homologue with 95% amino acid identity over the full length thereof, or any fragment thereof.

In one embodiment of the methods of the first aspect, said improved APP processing modulator is a modulator, particularly an inhibitor or stabilizer, of APP-CTF production, more particularly a modulator, particularly an inhibitor or stabilizer, of g-secretase activity. In a particular embodiment, said improved APP processing modulator is a therapeutic candidate to treat neurodegenerative disorders, particularly neurodegenerative diseases wherein the lysosomal function is compromised for example in Niemann Pick disease type C, more particularly the neurodegenerative disorder is AD. In another embodiment, the collection of known APP processing modulators is a collection of g-secretase modulators, more particularly g-secretase stabilizers and/or inhibitors.

In a second aspect, the therapeutic candidate is selected from a collection of test compounds, more particularly a library of biological and/or chemical compounds. Non-limiting examples of said library can be a library comprising small molecules, compounds with known functions, FDA approved drugs, compounds pre-screened on bioactivity or can be a drug repurposing library.

In some instances, the therapeutic candidate is selected using a “high content screening” (HCS) method that uses a series of experiments as the basis for high throughput compound discovery. Typically, HCS is an automated system to enhance the throughput of the screening process. However, the present invention is not limited to the speed or automation of the screening process. The method is neither limited to large or high-throughput or any scale, and can be refined based on the availability of test compounds or other variable features of the screening assay.

Methods are herein provided of screening for APP processing modulators, more particularly for therapeutic candidates to treat neurodegenerative disorders, particularly neurodegenerative disorders wherein lysosomal function is compromised, more particularly AD, comprising the steps of:
a) providing one or more cells; and
b) quantifying before and after administering a test compound to the one or more cells the level of APP-CTF in said one or more cells and/or the Ca2+ level in the lysosomes of said one or more cells and/or the cholesterol level in the lysosomes of said one or more cells;
   and identifying said test compound as an APP processing modulator if after the administration in step b) there is an at most 50%, at most 40%, at most 30%, at most 25%, at most 20%, at most 15%, at most 10% or at most 5% or not statistically significantly increased APP-CTF level and/or an at most 20%, at most 15%, at most 10% or at most 5% or not statistically significantly decrease in lysosomal Ca2+ level and/or an at most 25%, at most 20%, at most 15%, at most 10% or at most 5% or not statistically significantly increase lysosomal cholesterol level after the administration of the test compound compared to before said administration. In one embodiment, said one or more cells express a gamma-secretase complex and comprising a (poly)peptide SEQ ID NO:1 or a homologue with 95% amino acid identity over the full length thereof, or any fragment thereof.

In one embodiment the method further comprises a step c) of quantifying Ab peptides with a length of 38, 40, 42 and/or 43 amino acids produced in said one or more cells before and after administering the test compound; and identifying said test compound as an APP processing modulator if after the administration in step c) there is a statistically significant increase in the ratio of Ab38/Ab42, Ab40/Ab42, Ab40/Ab43 or of Ab (38+40)/Ab (42+43) compared to the situation before administering said test compound.

The different steps in the above methods are referred to as steps b) and c) for clarity. The methods of the second aspect do not require that step b) is executed prior to step c). Step c) of quantifying Ab peptides can be executed first and thereafter can the actions described in step b) be executed. The order of step b) and c) is thus not relevant.

In a particular embodiment of all methods herein disclosed, the one or more cells expressing a gamma-secretase complex and a (poly)peptide SEQ ID No. 1 or a homologue with 95% amino acid identity over the full length thereof, further comprise an abnormal level of APP-CTF. Said abnormal level which can be defined as an statistically significantly higher level of APP-CTF compared to a wild-type cell, can be obtained due to PSEN or APP mutations that destabilize g-secretase/Ab complexes as those described in Szaruga et al Cell 2017 or FAD-causing APP mutations as those described in Szaruga et al Cell 2017.

### Detection and Quantification Means

The methods described in the first and second aspect comprise steps in which different proteins, metabolites, signalling molecules, . . . are detected and quantified.

### APP-CTF

In one embodiment, the APP-CTF level is quantified via immune-based assays. In another embodiment, APP-CTF is fluorescently labelled and the APP-CTF level corresponds to the APP-CTF fluorescent signal. Measuring and quantified fluorescent signal in one or more cells is well established in the field.

### Ab Fragments

“Quantification” of the Ab peptides produced in the one or more cells herein disclosed means that several types and lengths of Ab peptides are detected or measured using a suitable method for said purpose, known by the person skilled in the art. Following the Ab peptide quantification, the final ratio of Ab38/Ab42 or of Ab40/Ab43 or of Ab40/Ab42 or of (Ab38+Ab40)/(Ab42+Ab43) can be easily calculated.

Detection and quantification of Ab peptides produced in one or more cells is in one embodiment obtained via “immune-based assays” or “immune-based detection” or “immune-based quantification”, used interchangeably herein, which refer to the most broadly used bio-detection technologies that are based on the use of antibodies, and are well known in the art. Antibodies are highly suited for detecting small quantities of specific peptides or proteins in the presence of a mixture of peptides or proteins. Said “immune-based detection” refers to a biochemical binding assay involving binding between antibodies and antigen, which measures the presence or concentration of a substance in a sample, such as a biological sample, or an in vitro sample, using the reaction of an antibody to its cognate antigen, for example the specific binding of an antibody to a specific Ab peptide. Both the presence of the antigen or the amount of the antigen present can be measured. Examples of immunoassays are enzyme linked immunosorbent assays (ELISAs), enzyme linked immunospot assay (ELISPOT), immunobead capture assays, Western blotting, gel-shift assays, protein arrays, multiplexed bead arrays, magnetic capture, fluorescence resonance energy transfer (FRET), a sandwich assay, a competitive assay, an immunoassay using a biosensor, an immunoprecipitation assay etc.

Antibodies are currently available to detect and distinguish each type of resulting Ab peptide (e.g. Ab38, Ab40, Ab42 and Ab43) relevant for determination of Ab ratios herein described. More information can be found in e.g. WO2018/130555A1.

Detection and quantification can also be performed using labels or tags. The term detectable label or tag, as used herein, refers to detectable labels or tags allowing the detection and/or quantification of the isolated peptides described herein, and is meant to include any labels/tags known in the art for these purposes. Particularly preferred, but not limiting, are affinity tags, such as chitin binding protein (CBP), maltose binding protein (MBP), glutathione-S-transferase (GST), poly (His) (e.g., 6x His or His6), Strep-tag®, Strep-tag II® and Twin-Strep-tag®; solubilizing tags, such as thioredoxin (TRX), poly(NANP) and SUMO; chromatography tags, such as a FLAG-tag; epitope tags, such as V5-tag, myc-tag and HA-tag; fluorescent labels or tags (i.e., fluorochromes/-phores), such as fluorescent proteins (e.g., GFP, YFP, RFP etc.) and fluorescent dyes (e.g., FITC, TRITC, coumarin and cyanine); luminescent labels or tags, such as luciferase; and (other) enzymatic labels (e.g., peroxidase, alkaline phosphatase, beta-galactosidase, urease or glucose oxidase). Also included are combinations of any of the foregoing labels or tags.

Detection and quantification of the Ab peptides produced in one or more cells is in another embodiment obtained via “mass-spectrometry” or “MS-based detection” or “mass-spectrometry-based quantification”, used interchangeably herein, which refer to detection/quantification methods specifically defining the desired Ab peptides, such as Ab38, Ab40, Ab42, Ab43. Examples of such MS-based quantification methods are provided in WO2018/130555A1, but also derived from Takami et al (2009 J Neurosci 29:13042-13052), and from Okochi et al (2013 Cell reports 3:42-51), the latter for instance applying Ab45 and Ab46 as substrates for gamma-secretase to follow the resulting cleavage products by MS. In another embodiment, the detection and quantification of said produced Ab peptides in said system comprises both, immune-based and MS-based techniques.

### Ca2+

Several methods for determining Ca2+ levels in cells or in cell organelles are available in the art. Calcium imaging is a microscopy technique to optically measure the calcium (Ca2+) status of an isolated cell, tissue or medium or in living animals. Calcium imaging takes advantage of calcium indicators or sensors, i.e. fluorescent molecules that respond to the binding of Ca2+ ions by fluorescence properties. Two main classes of calcium indicators exist: chemical indicators and genetically encoded calcium indicators (GECI).

Chemical indicators are small molecules that can chelate calcium ions. All these molecules are based on an EGTA homologue called BAPTA, with high selectivity for calcium (Ca2+) ions versus magnesium (Mg2+) ions. Binding of a Ca2+ ion to a fluorescent indicator molecule leads to either an increase in quantum yield of fluorescence or emission/excitation wavelength shift. Non-limiting examples are fura-2, indo-1, fluo-3, fluo-4, Calcium Green-1. In one embodiment, the lysosomal Ca2+ dynamics (i.e. both storage and release) can be monitored using ratiometric Ca2+ dyes (such as Fura-2AM). In another embodiment, said monitoring occurs in the presence of lysosomotropic agents such as glycyl-L-phenylalanine 2-naphthylamide (GPN) (Jadot et al 1984; Morgan et al 2020; Yuan et al 2021) or L-leucyl-L-leucine methyl ester (LLOMe) (Thiele et al 1990; Morgan et al 2020). These peptides are thought to be cleaved by lysosomal proteases, causing an osmotic lysis of the lysosomes, and the release of their Ca2+ content.

Genetically encodable calcium indicators (GECIs) are powerful tools useful for in vivo imaging of cellular, developmental, and physiological process. GECIs do not need to be loaded into cells; instead the genes encoding for these proteins can be easily transfected to cell lines. It is also possible to create transgenic animals expressing the dye in all cells or selectively in certain cellular subtypes. Also GECIs inevitably rely on fluorescent proteins as reporters, including green fluorescent protein GFP and its variants (eGFP, YFP, CFP). In most cases GECIs are based on the Ca2+ binding protein Calmodulin (CaM). Non-limiting examples are camgaroos, G-CaMP, cameleons.

Hence, in one embodiment, the Ca2+ level in the lysosomes is determined by an image-based assay. In a particular embodiment, the Ca2+ level is determined by GCaMP6.

### Cholesterol

Several staining methods to detect and quantify intracellular cholesterol levels are available in the art.

The use of fluorescent probes that specifically bind membrane cholesterol allows the visualization and imaging of cellular cholesterol. A non-limiting example is the GFP-D4 probe which is a fusion between the fluorescent protein GFP and the D4 fragment of perfringolysin O, a pore-forming toxin from the bacterium Clostridium perfringens. Perfringolysin O is a cholesterol-dependent cytolysin secreted as a water-soluble monomer that recognizes and binds cholesterol-rich membranes where it oligomerizes and creates pores. An alternative is GST-D4H*-mCherry as used herein and described in Davis et al (2021 Dev Cell). The D4 fragment of perfringolysin O is able to bind cholesterol-rich membranes (>30 mol % cholesterol) but is devoid of pore-forming activity (Wilhelm et al 2019). Interestingly, mutants of GFP-D4, able to bind membrane cholesterol with higher sensitivity, have been generated.

Another non-limiting example is filipin, a polyene macrolide extracted from the bacterium Streptomyces filipinensis. Filipin is naturally fluorescent and specifically binds to sterols. Filipin staining is a generally accepted tool for detection of cholesterol deposits, e.g. in the lumen of lysosomes.

Alternatively, cholesterol content can be estimated after lysosomal purification using superparamagnetic iron oxide nanoparticles (SPIONs). LE/Lys isolation can be achieved in high yield and purity by a magnetic affinity approach based on DMSA-coated SPIONs (Tharkeshwar et al 2017). Isolated fractions are then prepared and analyzed using shotgun lipid profiling (as in Tharkeshwar et al 2017).

Cholesterol levels can also be determined using the Amplex-Red cholesterol assay (Molecular Probes). This fluorometric assay based on an enzyme-coupled reaction detects both free cholesterol and cholesteryl esters. Cholesteryl esters are first hydrolyzed by cholesterol esterase into cholesterol, which is then oxidized by cholesterol oxidase to generate H2O2 and the corresponding ketone product. The Amplex Red reagent detects the generated H2O2 and in the presence of HRP produce the fluorescent resorufin.

In one embodiment, the cholesterol level in the lysosomes is determined by an image-based assay. In a particular embodiment, the cholesterol level in lysosomes is determined by filipin staining or by a fluorescently labelled D4 or D4H* probe.

### Intracellular Compartments

To visualise, detect and/or localise the different compartment of the endocytic pathway several markers are available in the art. For example, and without the intention for being limited, the LE/Lys compartment can be detected using LAMP1 (as provided in the Examples) or by Lysotracker, the EE can be detected using EEA1 (as provided in the Examples), the ER can be detected by Sec61 (as provided in the Examples), while VPS35 is a marker for the endosomal recycling compartment (as provided in the Examples).

With respect to membrane contact sites, in between different organelles, a comprehensive list of markers can be found in Huang et al 2020. To investigate the membrane contact sites between the ER and either lysosomes or mitochondria, Sec-61 (ER marker) can be combined with either Lysotracker or LAMP1 (LE/Lys marker) or mitotracker red (mitochondria marker). More selective marker proteins for ER-lysosomes contacts include STARD3 and Rab7 on lysosomes and VAPB and Reticulon3 on the ER. Visualization of these marker proteins occurs through specific antibodies (and indirect immunofluorescence imaging) or through the transient expression of cDNA encoding these proteins fused to a fluorescent protein.

In one embodiment, the lysosomes are visualised by LAMP1 detection.

In yet another embodiment, the one or more cells used in the methods of current document are mammalian cells.

### Other Definitions

“Homologue” or “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. As disclosed herein, the degree of amino acid identity between a given reference amino acid sequence or fragment thereof and an amino acid sequence which is a variant or mutant of said given amino acid sequence or said fragment thereof will preferably be at least about 95%, 96%, 97%, 98%, or 99%. The degree of identity is given preferably for an amino acid region which is at least about 90% or about 100% of the entire length of the reference amino acid sequence. For example, if the reference amino acid sequence consists of 200 amino acids, the degree of identity is given preferably for at least about 180, or about 200 amino acids, preferably continuous amino acids. In particular embodiments, the degree/percentage of identity is given for the entire length of the reference amino acid sequence. In other embodiments, said fragments of the reference sequence with a degree of identity is referring to the degree/percentage of identity for said fragment wherein said fragment is aligned to the most optimally aligned region over the window of comparison of said reference sequence.

The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. The alignment for determining sequence identity, can be done with art known tools, preferably using the best sequence alignment, for example, using CLC main Workbench (CLC bio) or Align, using standard settings, preferably EMBOSS::needle, Matrix: Blosum62, Gap Open 10.0, Gap Extend 0.5.

“Compound” or “test compound” means any chemical or biological compound, including simple or complex organic and inorganic molecules, peptides, peptidomimetics, proteins, antibodies, carbohydrates, nucleic acids or derivatives thereof. The term “compound” is used herein in the context of a “drug candidate compound” or a “candidate compound for lead optimization” in therapeutics, described as identified with the screening methods herein disclosed.

The term “small molecule compound”, as used herein, refers to a low molecular weight (e.g., <900 Da or 40<500 Da) organic compound. The compounds also include polynucleotides, lipids or hormone analogues that are characterized by low molecular weights. Other biopolymeric organic test compounds include small peptides or peptide-like molecules (peptidomimetics) comprising from about 2 to about 40 amino acids and larger polypeptides comprising from about 40 to about 500 amino acids, such as antibodies or antibody conjugates.

### Therapeutic Applications

In a third aspect, the findings disclosed herein also substantiate therapeutic interventions. Hence, also provided is a cholesterol homeostasis regulator, particularly a stimulator or enhancer of cholesterol efflux from the late endosome and/or lysosome in a cell for use to treat neurodegenerative diseases, more particularly Alzheimer's disease.

In human cells the cholesterol esters (CE) reach the interior of the lysosome by means of LDL. The lysosomal acid lipase cleaves free fatty acids (FFA) from the CE. FFAs cross the lysosome membrane into the cytoplasm. Free cholesterol (CH) is captured by the NPC2 protein and transported to the NPC1 protein localized in the lysosomal membrane. The NPC1 protein receives CH from NPC2 and transports it across the lysosomal membrane into the cytoplasm, where CH can then be reintroduced into the metabolic pathway. When NPC1 or NPC2 are not produced or not functional, it leads to lipid accumulation inside the lysosomes. Dysregulation of NPC1/2 has been reported in the development of Niemann-Pick type C disease, a rare autosomal recessive disease characterized by abnormal accumulation of cholesterol and sphingolipids in endolysosomal compartments that results in neurological dysfunction and liver and lung failure (Hoque et al 2020; Sitarska et al 2021).

In one embodiment, said stimulator or enhancer of cholesterol efflux from the late endosome and/or lysosome is a genetic or molecular stimulator or enhancer. In a particular embodiment, said stimulator or enhancer is a transgene, more particularly said transgene is NPC1 and/or NPC2.

In one embodiment, the NPC1 transgene as used herein encodes the human NPC intracellular cholesterol transporter 1 depicted in SEQ ID No. 8. In another embodiment, the NPC2 transgene as used herein encodes the human NPC intracellular cholesterol transporter 2 depicted in SEQ ID NO: 9 or 10.

The stimulator or enhancer of cholesterol efflux from the late endosome and/or lysosome can be expressed from recombinant circular or linear DNA plasmids using any suitable promoter. Suitable promoters for expressing these stimulators or enhancers from a plasmid include, for example, the U6 or H1 RNA pol III promoter sequences and the cytomegalovirus promoter. Selection of other suitable promoters is within the skill in the art. Non-limiting examples are neuronal-specific promoters, glial cell specific promoters, the human synapsin 1 gene promoter, the Hb9 promotor or the promoters disclosed in U.S. Pat. No. 7,341,847B2.

The recombinant plasmids can also comprise inducible or regulatable promoters for expression of the stimulator or enhancer in a particular tissue or in a particular intracellular environment. NPC1 and/or NPC2 expressed from recombinant plasmids can either be isolated from cultured cell expression systems by standard techniques, or can be expressed intracellularly, e.g. in brain tissue or in neurons. NPC1/2 can also be expressed intracellularly from recombinant viral vectors.

Administration of NPC1 and/or NPC2 comprises transduction, such as viral transduction. In a further embodiment, administration of NPC1 and/or NCP2 comprises adeno-associated virus transduction. In one embodiment, transduction of NPC1 and/or NCP2 utilises a viral vector which specifically targets or infects the cells of the tissue or organ of interest. In a particular embodiment, transduction comprises a viral vector capable of crossing the blood-brain barrier. In one embodiment, transduction comprises a blood-brain barrier crossing adeno-associated virus. Thus, in one embodiment, transduction comprises a neurotropic virus or viral vector. In another embodiment, the viral vector is a neurotropic virus or viral vector. Examples of neurotropic viruses and viral vectors capable of crossing the blood-brain barrier include, but are not limited to, AAVrh.8, AAVrhIO and AAV9 as well as its variants and derivatives (e.g. AAVhu68 and PHP.B). In certain embodiments, the NPC1 and NPC2 are comprised in a viral vector, such as a neurotropic virus or viral vector and/or an adeno-associated virus vector. In a further embodiment, transduction comprises the adeno-associated virus variant AAV9 and its derivatives, such as PHP.B. In a yet further embodiment, transduction comprises a PHP.B viral vector. In another embodiment, NPC1 and/or NPC2 are comprised in a PHP.B viral vector. Viral vectors may be used to integrate the target sequence, such as the NPC/and/or NPC2 transgene, into the host cell genome, such as the genome of a cell of the tissue or organ of interest. Viral vectors, such as neurotropic viruses or viral vectors and adeno-associated viral vectors, may also be used to enable stable or long-term expression without integration of the target sequence into the host cell genome.

Administration of a recombinant plasmid comprising NPC1 and/or NCP2 as defined herein can also be done directly to the tissue or organ of interest. Examples of direct administration include injection directly into the tissue or organ of interest, such as by intracranial injection, or utilise a suitable delivery device.

In another embodiment, said cholesterol homeostasis regulator or stimulator or enhancer of cholesterol efflux from the late endosome and/or lysosome is a chemical or pharmaceutical compound. A non-limiting example of the cholesterol homeostasis regulator is Miglustat (C10H21NO4; CAS nr 72599-27-0) also known as OGT 918 and N-butyl-deoxynojirimycin and sold under the brand name Zavesca. Miglustat is an iminosugar that acts as an inhibitor of glucosylceramide synthase needed in the early stages of glycosphingolipid synthesis. This compound was approved by the Food and Drug Administration (FDA) for the treatment of Gaucher disease and is able to cross the blood-brain barrier (BBB). Oral administration in Npc1 mice reduced the accumulation of gangliosides in the brain, slowed the neurological progression of the disease, and increased lifespan by approximately 33% (Zervas et al 2001). Subsequent studies on cats showed a reduced accumulation of gangliosides in the brain, delayed onset of neurological symptoms and increased lifespan by approximately 74% (Zervas et al 2001). Due to the promising results of animal studies, clinical trials were started in NPC patients in 2002 (clinicaltrials.gov). They showed neurological improvement or stabilization (e.g. dysphagia, supranuclear gaze palsy) (Pineda et al 2019; Patterson et al 2007; Wraith et al 2010).

Another non-limiting example of a stimulator or enhancer of cholesterol efflux from the late endosome and/or lysosome in a cell that is herein provided for use to treat Alzheimer's disease, is HP-beta-CD (2-Hydroxypropyl-beta-cyclodextrin). HP-beta-CD (C54H102039; CAS nr 94035-0.-6/128446-35-5) is a cyclic oligosaccharide with a hydrophobic interior. It is initially absorbed into the endolysosome where it transports unesterified cholesterol to the cytosol and reduces its accumulation in the endolysosomes independently of NPC1 and NPC2 proteins. Unfortunately, HP-beta-CD is not able to cross the BBB when administered systemically, possibly because of its large size. Hence, it should be administered intrathecally or intracranially or fused to a BBB transporting moiety. Treating Npc1 cats every 2 weeks with an intrathecal dose of 120 mg HP-beta-CD reduced neurological dysfunction and lipid accumulation in neurons (Vite et al 2015). HP-beta-CD is currently one of the most promising NPC therapeutic agents in clinical trials. Also HP-beta-CD based compounds are envisaged herein, for example polyrotaxanes comprising beta-CDs threaded along a polymer chain capped with terminal bulky molecules as disclosed in Tamura and Yui (2014 Sci Reports 4) or pluronic/beta-CD-based polyrotaxanes comprising terminal disulfide linkages that can release threaded beta-CDs in lysosomes (Tamura and Yui 2014).

Also HP-gamma-CD (C72H128048; CAS nr 128446-34-4) was shown to reduce intracellular free cholesterol levels and normalize the lysosome changes in Npc1-null cells (Hoque et al 2020). Hence, in a particular embodiment, said stimulator or enhancer of cholesterol efflux from the late endosome and/or lysosome in a cell is 2-hydroxypropyl-beta-cyclodextrin or 2-hydroxypropyl-gamma-cyclodextrin.

Another non-limiting example is vorinostat (C14H20N203; CAS nr 149647-78-9). Vorinostat is an inhibitor of histone deacetylases that reduces the accumulation of lipids in the lysosomes of cultured skin fibroblasts of NPC patients. It is also known as suberanilohydroxamic acid (suberoyl+anilide+hydroxamic acid abbreviated as SAHA) and marketed under the name Zolinza.

Also other compounds that inhibit histone deacetylase 1, 2 and/or 3 are herein provided as stimulator of cholesterol efflux from the late endosome and/or lysosome in a cell for use to treat Alzheimer's disease. Valproic acid, a weak histone deacetylase inhibitor (HDACi), had been shown to have some benefit in NPC1−/− neural stem cells (Kim et al 2007 Biochem Biophys Res Commun 360). Broad spectrum HDACi such as trichostatin A (TSA), LBH589, Chidamide, AR427 also efficiently correct cholesterol accumulation in NPC disease models (Cruz et al 2021 ACS Pharmacol Transl Sci) and are thus envisaged herein for use to treat AD.

Recently, it was demonstrated that HSP90 inhibition by administration of HSP90 inhibitors 17-AAG, AUY922, Ganetespib, AT13387, SNX2112 or TAS-116 resulted in clearance of cholesterol from LE/Ly (Pipalia et al 2021 bioRxiv). HSP90 inhibition led to an increase in HSP70 expression and interestingly HSP70 overexpression mimicked the HSP90 inhibitory effect on cholesterol clearance (Pipalia et al 2021 bioRxiv). The same effect could be observed by administration of arimoclomol (C14H20CIN303; CAS nr 289893-25-0). Arimoclomol is a hydroxamic acid derivative of bimoclomol that amplifies Heat Schock Protein 70 (HSP70) gene expression (Hargitai et al 2003 Biochem Biophys Res Commun 307).

Another non-limiting example of a chemical compound envisaged herein for use to treat AD by correcting lysosomal cholesterol levels is genistein. Genistein was shown to increase lysosomal exocytosis with a decrease in cholesterol accumulation as result (Argüello et al 2021 Int J Mol Sci 22).

Hence, in one embodiment, the stimulator or enhancer of cholesterol efflux from the late endosome and/or lysosome in a cell as provided herein for use to treat Alzheimer's disease is miglustat, 2-hydroxypropyl-beta-cyclodextrin, 2-hydroxypropyl-gamma-cyclodextrin, 2-hydroxypropyl-beta-cyclodextrin-based polyrotaxanes, vorinostat, HDAC1/2/3 inhibitor (particularly valproic acid, trichostatin A, LBH589, Chidamide or AR427), HSP90 inhibitor (particularly 17-AAG, AUY922, Ganetespib, AT13387, SNX2112 or TAS-116), arimoclomol or genistein.

### EXAMPLES

### Example 1: Chronic Inhibition of G-Secretase Induces Lysosomal Dysfunction Prior to Affecting Endosomes

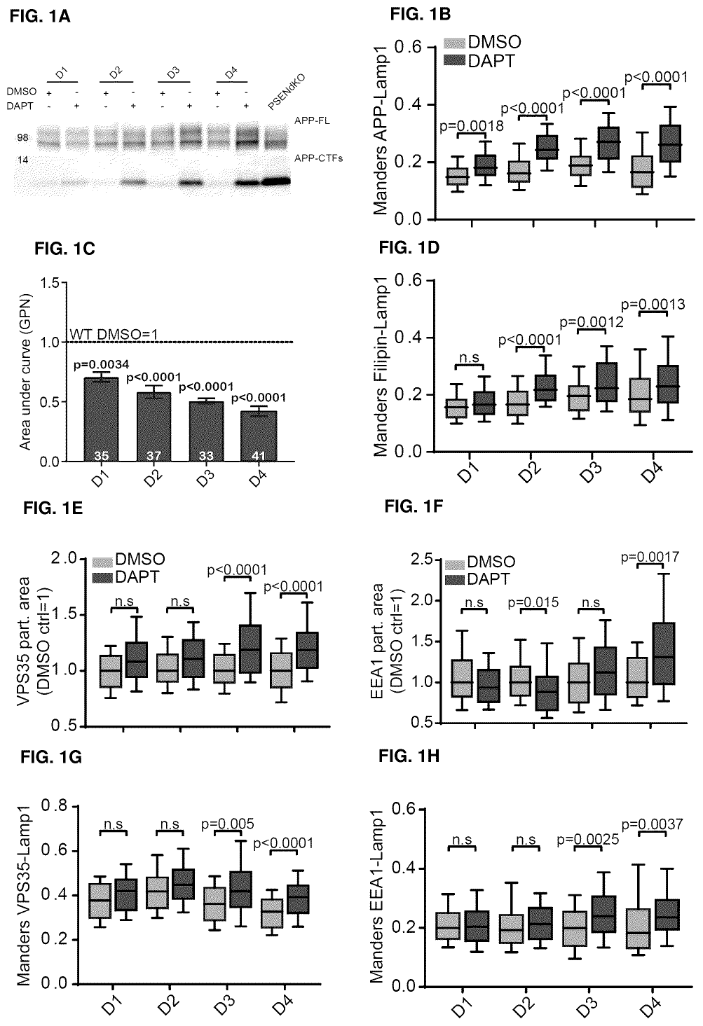

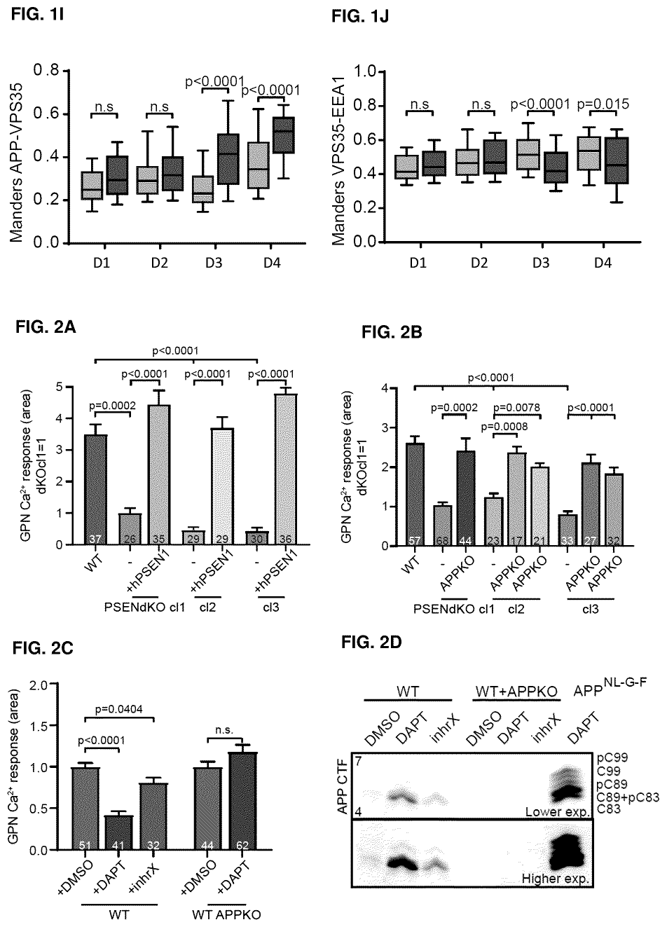

To address the sequence of events leading to a broad failure of the endosomal to lysosomal system, we treated wild-type (WT) mouse embryonic fibroblasts (MEFs) with the g-secretase inhibitor, DAPT, for 4 days (1 μu). DAPT-treated cells rapidly accumulated APP-CTFs (FIG. 1A) and an increasing APP-CTF accumulation was observed in LAMP1-positive LE/Lys (FIG. 1B). This accumulation strongly correlated with a significantly reduced lysosomal Ca2+ content, evaluated in Fura2-AM loaded cells challenged with Gly-Phe-b-naphtylamide (GPN) (Jadot et al 1984; Morgan et al 2020; Yuan et al 2021) (FIG. 1C).

In addition, DAPT-treated cells started to accumulate cholesterol in LE/Lys, however only significantly from day 2 (FIG. 1D). When investigating endosomal compartments, VPS35-positive organelles were found to be increased (FIG. 1E), underscoring a potential defect in endosomal recycling; however, only following 3 days of DAPT treatment and thus downstream of lysosomal dysfunctions. Importantly, early endosomes, as identified through Early endosome antigen-1 (EEA1), became significantly enlarged only after 4 days of treatment, indicating a late event (FIG. 1F).

Increased particle size coincided with increased colocalization of VPS35 and EEA1 with Lamp1, suggesting a general delay in endosome to lysosome maturation and a partial collapse of early and late endosomal compartments in DAPT-treated cells (FIG. 1G-H). This is further corroborated by increased co-localization APP-CTFs in collapsing early to late and recycling endosomes (FIG. 1B, H, I, J). Hence, these data support a chronology in which lysosomal dysfunction lies at the root of the endolysosomal collapse.

### Example 2: Knocking-out APP in PSENdKO Cells Corrects Endolysosomal Dysfunctions

Given the direct link between APP processing and endolysosomal defects, we decided to decipher the mechanistic role of APP proteolysis and, in particular, APP-CTFs. Independent PSENdKO MEF clones (Escamilla-Ayala et al 2020), were stably rescued with human PSEN1 (hPSEN1), using retroviral transduction.

Nicastrin (NCT) maturation was restored as well as PEN2 stabilisation, PSEN1 endoproteolysis and APP-CTFs clearance, indicating that PSEN1/g-secretase activities were rescued to WT levels (data not shown). As for DAPT-treated cells, deficiency of PSEN expression and thus g-secretase complex formation, resulted in a significant reduced lysosomal Ca2+ content (FIG. 2A). Only a concomitant minor alkalinisation was found. No defects were observed in the N-glycosylation of endogenous V0a1 subunit of the V-ATPase, nor in V-ATPase subunit levels in isolated lysosomes from WT, PSENdKO and hPSEN1-rescued MEFs, including the ratio of V0 over V1 subunits (data not shown). Only full alkalinization, using bafilomycin1 (Baf1), resulted in an equally affected lysosomal Ca2+ content as observed in PSENdKO cells. Together, these data strongly argue in favor of lysosomal Ca2+ defects being a primary cause of lysosomal dysfunctions in PSENdKO cells.

Both WT and PSENdKO MEFs were further gene-edited to knock-out APP, ensuring us with isogenic backgrounds. Depleting APP was sufficient to fully rescue the lysosomal Ca2+ defect caused by PSEN deficiency in independent clones, equally as observed in hPSEN1-rescued cells (FIG. 2A-B). Conversely, in the absence of APP expression, treatment with DAPT (1 μM, 4 days) did not affect the GPN-elicited Ca2+ response (FIG. 2C-D). This further supports the notion of APP playing a direct role in the control and maintenance of lysosomal Ca2+ homeostasis and, thus, being involved early on the endolysosomal collapse cascade.

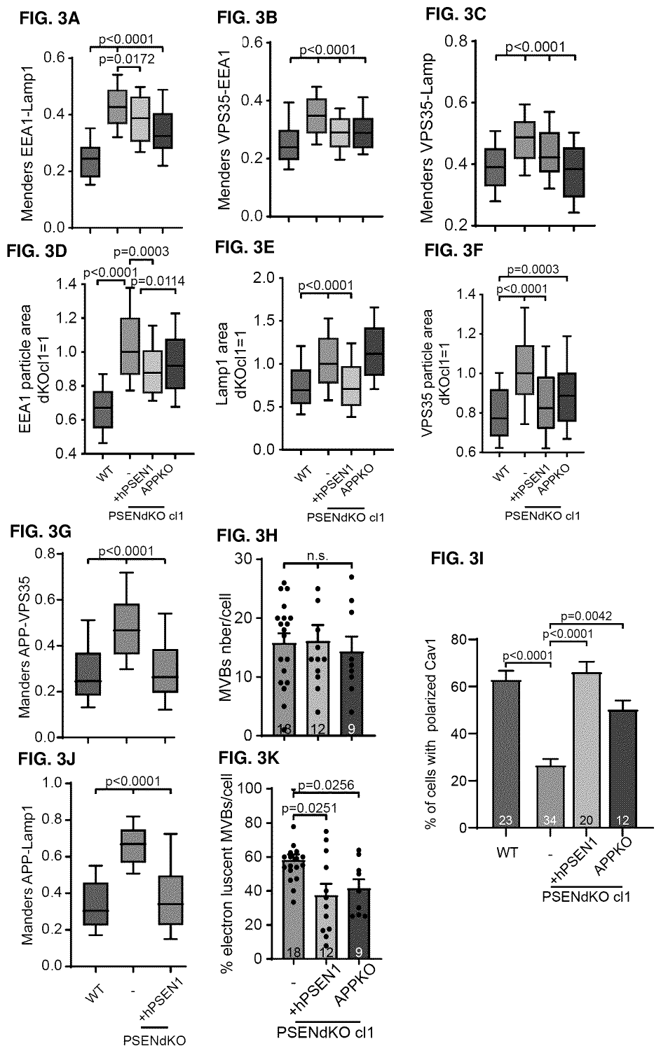

PSENdKO cells displayed enlarged LAMP1- and EEA1-positive compartments as well as an increased recruitment of VPS35 to these organelles (FIG. 3D-F). In agreement with our observations in DAPT treated cells, the significantly increased co-localization of EEA1-Lamp1 (FIG. 3A), VPS35-EEA1 (FIG. 3B) and VPS35-Lamp1 positive structures (FIG. 3C) supports a general delay/collapse in endosomal maturation. VPS35 levels were maintained between cell lines, excluding the enlargements to be grounded in an altered retromer expression. Of note, both VPS35-positive and Lamp1-positive structures in PSENdKO cells accumulate APP-CTFs, an accumulation relieved when hPSEN1 is reintroduced (FIG. 3G, J).

Re-expression of hPSEN1 as well as depletion of APP reduced the size of EEA1-positive and VPS35-positive organelles, and decreased the co-localization of endosomal markers, thus alleviating the delay in endolysosomal maturation (FIG. 3A-D, F). On the ultrastructural level, transmission EM (TEM) revealed enlarged multivesicular bodies (MVBs) with fewer intraluminal vesicles in PSENdKO as compared to hPSEN1 WT as well as APP depleted cells (FIG. 3H, K). This is consistent with a maturation delay and with immature MVBs accumulating at an early stage in the degradative pathway, i.e. at the crossroad of recycling and degradation (Huotari and Helenius, 2011). Of note, APP depletion did not revert the increased lysosomal area back to WT, in agreement with APP being required for the proper maintenance of endolysosomal size (Kessissoglou et al 2020).

In addition to endolysosomal defects, the trafficking of raft-associated components such as GM1 gangliosides (evidenced by CtxB Alexa-488) and caveolin-1 (Cav1) was altered upon PSEN depletion. When cells were brought to suspension, surface-localized CTxB and Cav1 rapidly internalized and relocated to a perinuclear compartment (Balasubramanian et al 2007), irrespective of the presence of PSEN1 and underscoring that endocytosis is not affected by PSEN deficiency (data not shown). Replating cells on fibronectin failed to restore the polarized distribution of CTxB and Cav1 at the cell surface in PSENdKO cells (FIG. 3I), whereas this was rescued by hPSEN1 reintroduction or APP depletion. This prolonged intracellular clustering in PSENdKO cells indicates therefore a defect in endosomal recycling.

Taken together, our data underscore that all major endosomal and lysosomal defects observed in PSENdKO cells can be traced back to APP expression. How APP mechanistically interferes in these processes is addressed in the following Examples.

### Example 3. Both APP a- and b-CTFs Induce Endolysosomal Defects

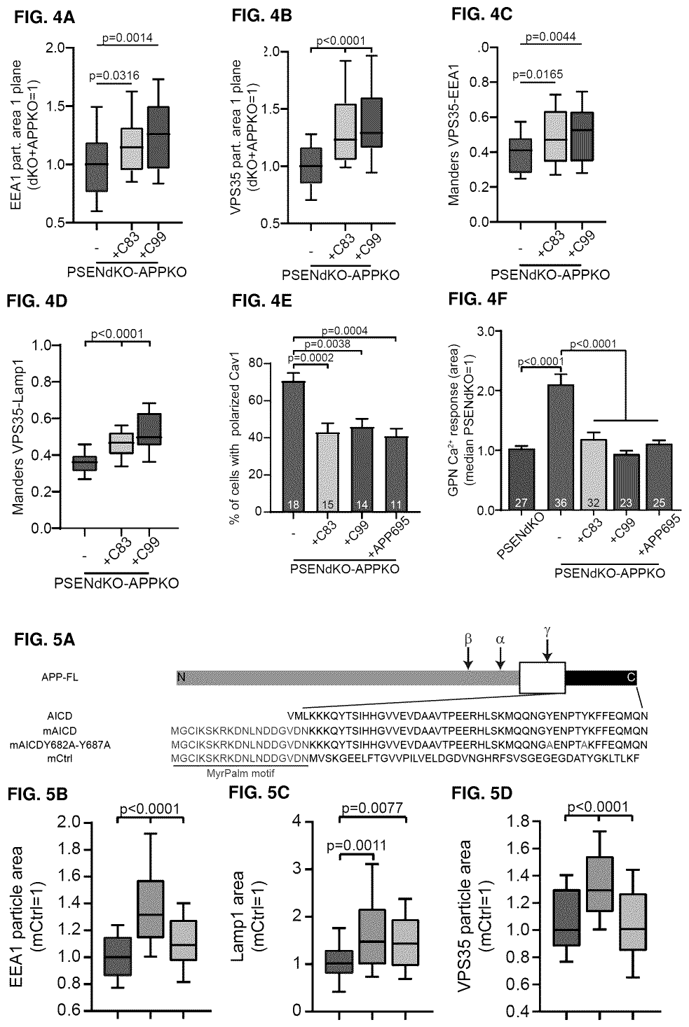

Until now APP-mediated neurotoxicity is thought to be only contributed by the combined b- and g-secretase activity. Based on the findings herein disclosed, we wondered whether a pathological effect can be observed of the APP-CTF produced by a- and g-secretase. We therefore stably re-introduced full-length APP695, a-CTFs (C83) or b-CTFs (C99) in PSENdKO-APPKO cells. We surprisingly found that both a- and b-CTFs equally resulted in the appearance of enlarged EEA1- and VPS35-positive compartments and in an increased co-localization of early and late endosomal markers (FIG. 4A-D). Thus, the endolysosomal collapse as seen in PSENdKO and DAPT-treated WT cells may arise to a similar extent from the accumulation of different types of APP-CTFs. Correspondingly, re-expression of APP a- or b-CTFs led to a defective endosomal recycling (FIG. 4E) and, like APP695, significantly impacted on lysosomal Ca2+ homeostasis, similar to PSENdKO cells (FIG. 4F).

### Example 4. Endolysosomal Defects Require the Membrane Anchoring of the APP Intracellular Domain

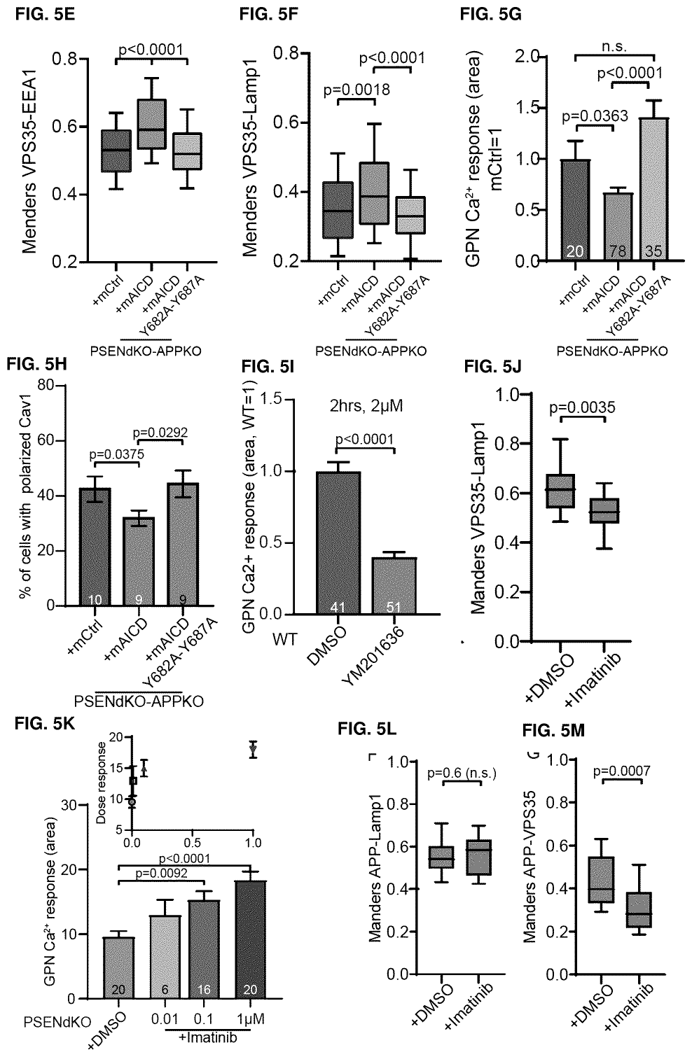

As we have shown that a different N-terminus of APP-CTFs is not a differentiating factor in the observed endolysosomal defects, we next focused on the APP intracellular domain (AICD; FIG. 5A). We stably expressed AICD modified at its N-terminus with a myristyolation motif (mAICD), ensuring membrane anchoring in our PSENdKO/APPKO cells (FIG. 5A).

Expression of mAICD fully recapitulated endolysosomal defects observed in PSENdKO as well as in APP-CTFs-rescued PSENdKO-APPKO cells. In particular, mAICD expression decreased lysosomal Ca2+ content (FIG. 5G), in enlarged EEA1-, VPS35- and LAMP1-positive compartments, along with an increased VPS35-EEA1 and VPS35-LAMP1 co-localization, showing a delay/collapse in endolysosomal maturation (FIG. 5B-F), as well as an endosomal recycling defect (FIG. 5H). Interestingly, stable expression of a mutant variant of mAICD with the Tyr residues of the YENPTY motif mutated to Ala, did not induce endolysosomal defects including lysosomal Ca2+ dyshomeostasis (FIG. 5B-H). These data support that the APP-CTF is the active signalling fragment of APP of which the levels need to be homeostatically controlled through g-secretase.

The YENPTY is a key motif for the binding of adaptor proteins such as SorLA (Andersen et al 2005), Fe65 (Borg et al 1996), X11/Mint (Borg et al 1996), Dab1 (Howell et al 1999) as well as PikFYVE (Currinn et al 2016), indicating a pleiomorphic nature of downstream signalling. Part of the effects could originate from the re-location of signalling-defective mAICD from a pronounced endolysosomal localization to a major residence at the cell surface (data not shown). Endolysosomal defects similar to those observed in PSENdKO cells are observed in cells treated with the PikFYVE inhibitor YM201636 (FIG. 5I). Likewise, inhibiting Abl kinase in PSENdKO cells, using Imatinib (Netzer et al 2003), alleviated the delay in endolysosomal maturation as evidenced from the lowered VPS35-LAMP1 co-localization (FIG. 5J) and restored dose-dependently lysosomal Ca2+ content (FIG. 5K). Improved sorting and recycling resulted in a significant decrease of APP-CTFs in VPS35-positive organelles (FIG. 5M), albeit this was not observed for LAMP1-positive LE/Lys (FIG. 5L). Altogether, this indicates that inhibition of signalling emanating from APP-derived fragments alleviates endolysosomal defects, mostly through restoration of cargo recycling.

### Example 5. Reduced Lysosomal Ca2+ Content Originates From an Altered Communication With the ER

It is herein disclosed that a decreased lysosomal Ca2+ content lies at the root of endolysosomal dyshomeostasis observed during chronic g-secretase inhibition. The data above suggest this may originate from a sustained signalling from membrane tethered AICD fragments. To decipher this, we first addressed the origin of the lysosomal Ca2+ dyshomeostasis.

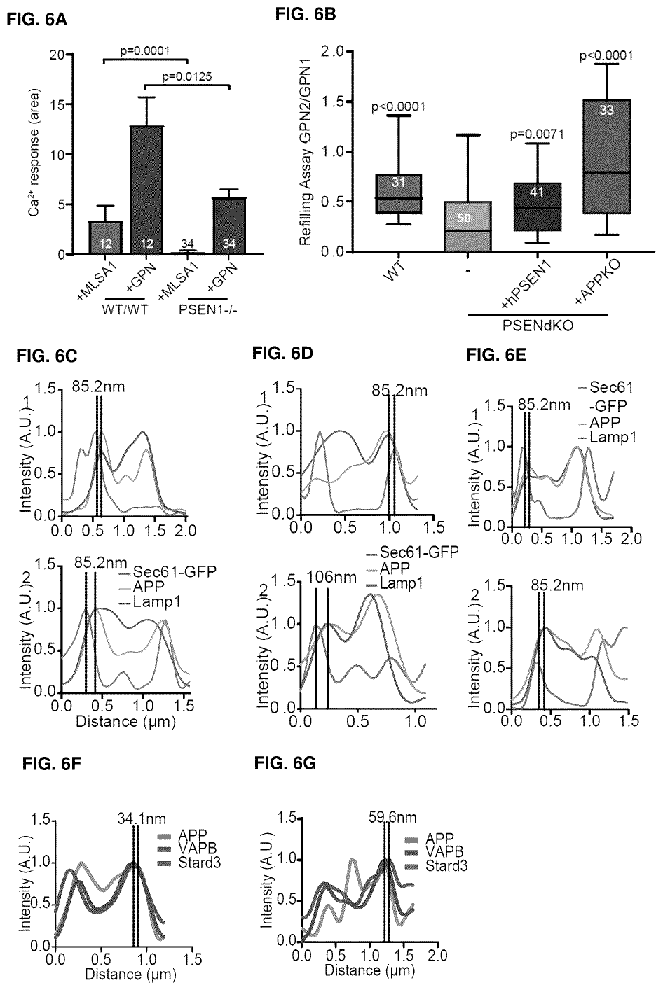

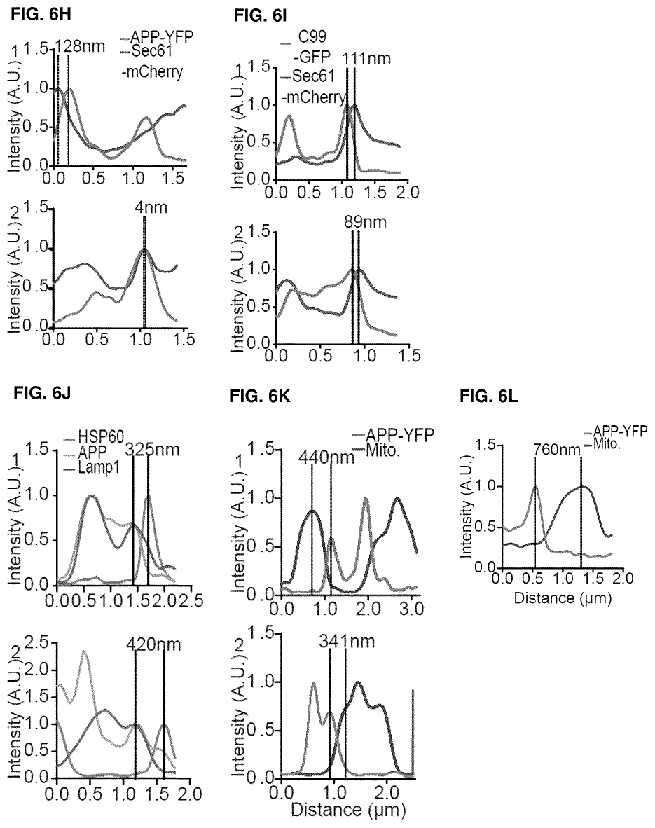

In contrast to previous reports (Lee et al 2015; Shen et al 2012), we could not relate this defect to an overactivation of the lysosomal Ca2+ channel TRPML1. Firstly, a challenge with the TRPML1 agonist MLSA1 did not reveal an overactivation of the channel in three independent PSENdKO clones (data not shown). Secondly, we generated a KI mouse model wherein TRMPL1 was endogenously tagged with the Ca2+ sensor Gcamp6S and crossed this line with our PSEN1KO mouse model. Primary neurons derived from PSEN1 KO/KO Gcamp6S-TRPML1 KI/KI embryos did not display an increased lysosomal Ca2+ response when challenged with MLSA1, in contrast to what was previously reported (Lee et al 2015), whereas the GPN elicited Ca2+ response remained reduced as compared to their WT/WT counterparts (FIG. 6A).

Another potential source of the lysosomal Ca2+ defect could originate from a failed communication between the ER and lysosomes. We therefore assessed the Ca2+ refilling of the lysosomes from the ER in our different cell lines. Fura2-AM loaded cells were repeatedly challenged with GPN in absence of extracellular Ca2+, allowing the emptying of lysosomal Ca2+, alternating with “rest phases” in the presence of extracellular Ca2+, to allow lysosomes to reseal and refill from the ER. PSENdKO cells displayed a significantly decreased lysosomal Ca2+ refilling, which was restored upon stable hPSEN1 re-expression or when APP was additionally knocked out (FIG. 6B).

These findings point to a derailed ER-to-lysosome Ca2+ transfer, which occurs through LE/Lys-ER MCSs (Burgoyne et al 2015; Garrity et al 2016; Yang et al 2019). We therefore investigated next whether APP-CTFs interfered within or near LE/Lys-ER MCSs. We performed super-resolution Airyscan imaging of PSENdKO and PSENdKO-APPKO cells stably rescued with APP-C99 or mAICD and co-transfected with Sec61-GFP (to mark the ER). APP-CTFs fragments often appeared clustered on the limiting membrane of LAMP1-positive LE/Lys and in close apposition to Sec61-GFP+ER membranes as shown by the respective line scans (FIG. 6C-E), indicating their localization in LE/Lys-ER MCSs. APP immunoreactivity did not overlap with Sec61-GFP, making APP-CTFs to exert their role in MCSs from the side of LE/Lys. Similar close appositions in the range of 80 to 120 nm, typical for MCSs, were observed in mAICD expressing cells (FIG. 6E).

To further validate this, we promoted the formation of LE/Lys-ER MCSs through overexpression of VAPB and Stard3 (Alpy et al 2013), leading to the expected prominent colocalization of APP/APP-related fragments with Stard3 in LE/Lys-ER MCSs (FIG. 6F-G). To further confirm the localization of APP-CTFs to these MCSs, we treated cells with a hypotonic (5% DMEM in water) solution, which results in the rapid vacuolization of intracellular compartments without affecting inter-organellar contacts (King et al., 2020). The formation of large intracellular vesicles (LICVs) makes MCSs more visible, facilitating co-localization analysis. Hypotonic treatment of PSENdKO-APPKO cells transfected with Sec61-mCherry and either APP-YFP (FIG. 6H) or C99-GFP (FIG. 6I), resulted in APP-positive LICVs contacting ER-derived LICVs and with APP/APP-CTFs being often enriched at these contact sites within a similar range of 80-120 nm to Sec61-mCherry+LICVs (FIG. 6C-D). These data therefore demonstrate the localization of APP-CTFs to LE/Lys-ER MCSs where it may modulate lysosome-to-ER Ca2+ transfer. These findings contradict reports associating APP-CTFs with mitochondria-associated membranes (MAMs), denoting a role in ER-mitochondria MCSs (Montesinos et al 2020; Pera et al 2017). However, line scans of triple immunostaining of PSENdKO and PSENdKO-APPKO cells rescued with C99 with HSP60 did not reveal close appositions with mitochondria reminiscent of MCSs (FIG. 6J). Likewise, live imaging of cells treated with hypotonic medium, showed much larger distances between APP- and mitotracker positive LICVs (Figure K-L), not supporting the presence of APP-C99 in mitochondria-ER MCSs.

### Example 6. Accumulation of Membrane-Tethered APP Fragments Affect LE/Lys-ER MCS Morphology and LE/Lys Dynamics

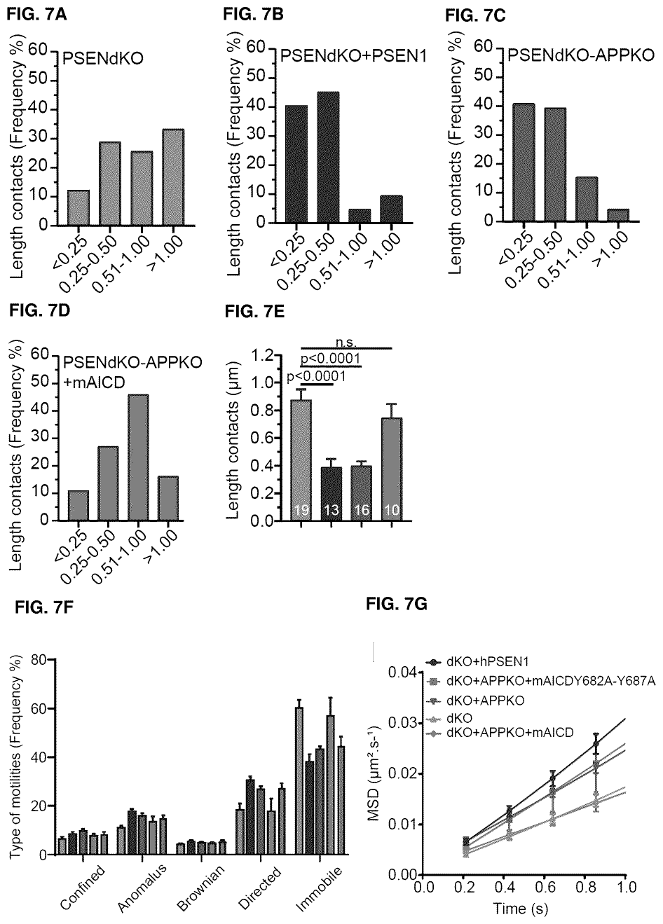

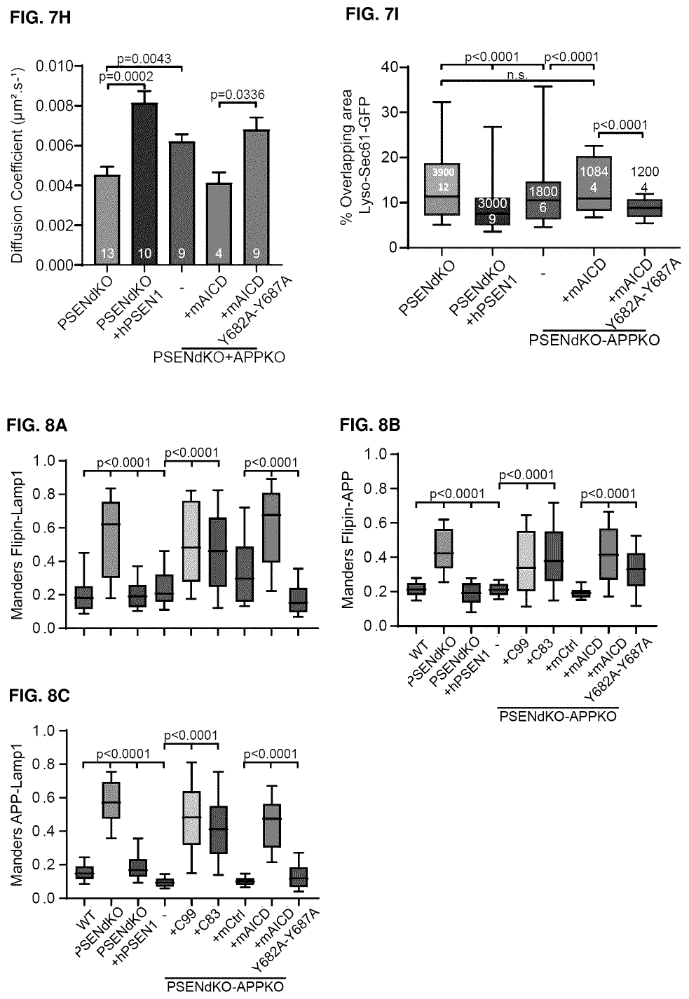

We next investigated whether the increased localization of APP-CTFs impacts on LE/Lys-ER MCS morphology. Visualization of the ER by transmission EM was facilitated by co-expressing HRP-myc-KDEL, followed by DAB treatment (Giordano et al 2013). Firstly, PSENdKO cells display increased numbers of electron-lucent vesicles (data not shown) likely reflecting a delayed maturation to MVBs. In line with the above, this phenotype disappeared in hPSEN1-rescued PSENdKO and PSENdKO-APPKO cells but re-appeared when the latter cells were stably rescued with mAICD. Whereas the HRP-reactive ER of both PSENdKO and PSENdKO-APPKO mAICD cells generally appeared more swollen, in addition, many enlarged contacts of ER were noticeable with LE/MVBs (FIG. 7A, D), sometimes largely encapsulating them. Reintroducing hPSEN1 in PSENdKO or depleting APP herein or introducing a mutant mAICD in PSENdKO-APPKO significantly reduced lengths (FIG. 7B-C, E), indicating a normalization of MCSs.

We next reasoned that the enlarged contacts of ER with LE/Lys might as well affect other aspects of LE/Lys, including motility. To test this, we used lattice structured illumination microscopy (SIM; Zeiss Elyra-7) and compared the dynamics of Lysotracker-labelled acidic organelles in the different cell lines. PALMtracer (see Experimental procedures) was used to track individual organelles and to quantify changes in the time-correlated mean-squared displacement (MSD). This approach allows to distinguish different types of motility from random motion (Escamilla-Ayala et al 2020). In agreement with the observed enlarged LE/Lys-ER contacts, only cells accumulating or expressing membrane tethered AICD fragments (either CTFs or mAICD) display a significantly increased proportion of immobile tracks, reminiscent of less motile organelles (FIG. 7F). In agreement, we observed a reduction in the MSD and diffusion coefficients (FIG. 7G-H). The decreased motility correlated with a longer retention of the Lysotracker Red-labelled organelles on the ER; indicated by the higher colocalization of these organelles (FIG. 7I).

### Example 7. Promoting Cholesterol Egress Largely Restores Endolysosomal Demise in PSEN Deficient Cells

We show herein that a decreased lysosomal Ca2+0 content of PSEN deficient cells can be functionally linked to the accumulation of APP-CTFs or mAICD converging with altered LE/Lys-ER MCSs.

To further explore the mechanisms that are at stake, several cell biological assay were performed. Interestingly, free cholesterol, as measured by Filipin staining, strongly accumulated in Lamp1-compartments of PSENdKO and PSENdKO-APPKO cells rescued with APP-CTFs or mAICD (FIG. 8A). Within these organelles, Filipin majorly co-localized with APP-CTFs and mAICD (FIG. 8B-C). Depleting APP in a PSENdKO background or re-expression mutant mAICD reverted the accumulations back to WT levels. Hence, we wondered whether alleviating the burden of cholesterol in LE/Lys could correct lysosomal Ca2+ defects. We treated PSENdKO cells with an inhibitor of OSBP and ORP4L (OSW-1), counteracting the sequestration of cholesterol from the ER to the lysosome (Burgett et al 2011). The treatment did not decrease lysosomal cholesterol staining nor did it rescue lysosomal Ca2+ dysfunction of PSENdKO cells (data not shown). This indicates that cholesterol accumulating in LE/Lys is not originating from an ER pool.

Alternatively, we induced egress of lysosomal cholesterol through overexpression of NPC1 or treatment with 2-hydroxypropyl-γ-cyclodextrin (HPγCD), a drug shown to reduce intracellular free cholesterol and to enhance autophagic activity through the regulation of ER-Lysosomes contact sites (Hoque et al 2020; Singhal et al 2020). Reduced filipin staining in Lamp1-positive LE/Lys was confirmed (FIG. 8E,I). Both NPC1 overexpression and HPγCD treatment strongly reduced the accumulation of membrane-tethered APP fragments in Lamp1-positive LE/Lys of PSENdKO cells (FIG. 8D,H), and fully restored lysosomal Ca2+ content in PSENdKO cells (FIG. 8L). Of note, these treatments also partially restored the endolysosomal maturation, as seen by the reduced EEA1 area (FIG. 8G,K) and decreased colocalization of VPS35 with either EEA1 (FIG. 8F) or Lamp1 (FIG. 8J).

In conclusion, treatments that promoted cholesterol efflux from the LE/Lysosomes, but not from the ER, relieved both lysosomal Ca2+ and endolysosomal defects.

### Experimental Procedures

### Antibodies and Reagents

Rabbit polyclonal antibodies (pAb) to PSEN2-CTF (B24 1/2000 for WB), APP (B63.1, 1/5000 for WB), and monoclonal antibody (mAb) to NCT (9C3, 1/7000 for WB) were described earlier (Annaert et al 1999; Esselens et al 2004; Raemaekers et al 2012; Spasic et al, 2007; Spasic et al 2006). The following antibodies were commercially obtained: rabbit pAb anti-PSEN1 NTF (ab71181, abcam, 1/3000 for WB), anti-PSEN1 CTF (ab24748, abcam, 1/1000 for WB), anti-PEN2 (ab18189, abcam, 1/1000 for WB), anti-M6PR (PA3-850, Thermo, 1/100 in immunofluorescence (IF)), anti-EEA1 (E4156, Sigma, 1/300 in IF, 1/250 for WB), anti-v-ATPase A1 (v0a1, H-140, sc-28801, Santa Cruz Biotechnology 1/1000 for WB), anti-LC3B (NB600-1384H, Novus Biological, 1/2000 for WB), anti-caveolin1 (610060, BD Biosciences, 1/200 in IF); goat pAB anti-VPS35 (ab10099, abcam, 1/250 in IF); mouse mAb anti-β actin (AC15, A5441, Sigma Aldrich, 1/10000 for WB), anti-V0d1 (ab56441, abcam, 1/1000 for WB), anti-V1b2 (sc-166122, Santa Cruz Biotechnology, 1/1000 for WB), anti-HSP60 (611562, BD transduction 1/200 in IF), anti-PtdIns (3,5)P2 (Z-P035-2-EC, Echelon, mobitec, 1/200 in IF), anti-VPS35 (sc-374372, Santa Cruz Biotechnology, 1/500 for WB), anti-human Aβ 82E1 (JP10323, Tecan, 1/100 in IF), anticaveolin1 (610407, BD Biosciences, 1/200 in IF), anti-Flag (F1804, Sigma, 1/500 in IF); rat mAb anti-Lamp1 (sc-19992, Santa Cruz, 1:300 in IF, 1/1000 for WB); rabbit mAb anti-APP (Y188, ab32136, abcam, 1/500 in IF). Peroxidase-conjugated secondary antibodies were purchased from Biorad (1/10000 for WB). Alexa-conjugated fluorescent secondary antibodies (Goat or Donkey, Alexa 488, 555 and 647) were from Life Technologies (1/1000 in IF). Lysotracker (Red DND-99 L7528, Deep Red L12492, used at 1/20000), Mitotracker (Deep Red, M22426, used at 500 nM), CtxB conjugated with Alexa dyes (-488 C34775, -647 C34778) and phalloidin conjugated with Alexa fluor (-488 A12379; -568 A12380 and -647 A22287) were purchased from ThermoFisher Scientific; Filipin III Streptomyces filipensis (F4767, 200 μg·ml−1) from Sigma.

### Mice

TRPML1-Gcamp6 mouse generation TRPML1-Gcamp6 mice were produced by Ingenious targeting laboratory, and were obtained using the CRISPR-Assisted Reporter Knock-in Targeting Vector Construction strategy. Briefly, a targeting vector bearing homology arms, Gcamp6 and a selection cassette (Neo) was designed, using conventional cloning method. Colony PCR was used to amplify both 5′ and 3′ homology arms with about 820 bp and 1.3 kb in length, respectively, from a positively identified C57BL/6 BAC clone (RP23-298P17); the GCAMP6s was fused at the ATG start site in exon 1 of the TRPML1gene and a Neo cassette was inserted downstream of the potential promoter sequence in intron 1-2. This combination was cloned in to the iTL cloning vector (\tilde{ }2.45 kb) derived from pSP72 (Promega). The validated targeting vector was electroporated in embryonic stem(ES) cells, and resulting clones were screened by PCR and Southern Blotting. Targeted clones were microinjected into Balb/c blastocysts, and resulting chimeras were mated to C57BL/6 WT mice to generate Germline Neo deleted mice, leaving only the Gcamp6 reporter inserted. PSEN1WT or KO-TRPML1 Gcamp6 mouse were obtained by crossing PSEN1 WT/KO TRPML1-Gamp6 KI/KI.

### Cell Culture and Primary Hippocampal Neuron Culture

Mouse embryonic fibroblasts (MEFs) were maintained in DMEM-F12 (Invitrogen) containing 10% FCS and maintained in a humidified chamber with 5% CO2 at 37° C. Primary hippocampal neuron culture were obtained as previously described (Coen et al 2012; Esselens et al 2004). Briefly, hippocampal neurons were derived from embryonic day 17 embryos from heterozygous crosses and were cocultured with a glial feeder layer to allow proper neuronal differentiation and polarization. After dissociation, cells from hippocampi of individual embryos were plated on poly-l-lysine-coated coverslips in minimal essential medium (MEM) supplemented with 10% (vol/vol) horse serum. Twenty hours after plating, culture medium was replaced by serum-free neurobasal medium supplemented with B27 (Gibco). 5-Fluoro-2-deoxyuridine (F0503, Sigma, 10 μM final) was added at DIV4, to prevent glial proliferation. Hippocampal neurons were maintained at 37° C. and 5% CO2.

### Drug Treatments

Cells were treated with the following drugs, usually for 4 days, unless otherwise stated: DAPT (2634/10, Tocris) 1 μM final; Inhibitor X (565771-250 μg, Calbiochem/Merck) 1 μM final; β-Secretase Inhibitor IV (565788, Calbiochem/Merck) 2 μM final, (2-Hydroxypropyl)-γ-cyclodextrin (H125-5G-I, Sigma Aldrich) 1 mM final; Adam 10 inhibitor G1254023X (SML0789, Sigma Aldrich) 3 μM final. For OSW-1 (30310-1, Sanbio/Cayman Chemical), cells were treated overnight with 10 nM. Diluent treatment alone (in most cases DMSO, water for the cyclodextrin) was used as vehicle control.

### Plasmids and Cell Transfection

Cells were transfected using Fugene HD (E2311, Promega), according to the manufacturer's protocol (ratio 4:1 for Fugene HD reagent: DNA was applied). The following plasmids were used: mCherry-Sec61B (Addgene plasmid #121160, (Ma and Mayr 2018)); pAc-GFPC1-Sec61beta (Addgene plasmid #15108, (Shibata et al 2008)); mCherry-VAPB (Addgene plasmid #108126, (Zewe et al 2018)); Flag-Stard3-WT (Di Mattia et al 2020); C99-GFP; APP-YFP (Stamer et al 2002); HRP-KDEL (as in Schikorski et al 2007). In all cases, assays were carried out 24 h after the transfection.

### Generation of Stable Cell Lines

KO cell lines: PSEN1 and 2 KO cell lines (PSENdKO) cell lines were established as described in (Escamilla-Ayala et al 2020). Briefly, the web-based CRISPR design tool was used to select the sequence to be targeted in mouse PSEN1 and PSEN2 (i.e. 5′-CAACGTTATCAAGTACCTCCCCGAA-3′ and 5′-CAACGTCCT GGGCGACCGTCGGGCC-3′, respectively). Oligo pairs encoding guide sequences (obtained from Integrated DNA Technologies, IDT) were annealed and ligated into the pX330 plasmid (Addgene) according to the Zhang's laboratory protocol (https://www.addgene.org/crispr/zhang/). MEF cells were then transfected with the pX330-PSEN1 and PX330-PSEN2, using Fugene HD (Promega), in accordance with the manufacturer protocol. Selection of PSENdKO clones was achieved through serial dilution and confirmed by WB analysis. Further depletion of APP in different PSENdKO clones was achieved through electroporation of RNP complexes. Single guide RNA (sgRNAs) were designed to either target exon 2 (5′-GTACCCACTGATGGCAACGC CGG-3′) or exon 3 (5′-ACGGTAAGGAATCACGATGTGGG-3′). The Neon Transfection System (ThermoFisher Scientific, one pulse, 1650 V, 20 ms) was used to electroporate 150 000 cells with 10 pmol of Cas 9 (IDT) and 20 pmol of single guide RNA (IDT). As above, cells were amplified and selection of independent APPKO clones was performed with serial dilutions and WB characterization. All clones of interest were sequenced using Sanger sequencing.

Rescue cell lines generation: Cell lines were stably rescued using retro- or lentiviral particles. For virus generation (either retro or lentivirus), HEK293T cells were transfected using FuGENE6 (Promega) according to the manufacturer's protocol. For retrovirus packaging, pMSCV expressing the gene of interest was cotransfected with the helper plasmid plk (Ecopac). Of note, cDNA of human WT PSEN1 were already cloned in the retroviral vector PMSCV*-puromycin (Takara bio Inc; Nyabi et al 2003 and in Coen et al 2012). For lentiviral production, particles were produced by co-transfecting the HEK293T cells with the plasmid of interest, pCMV-ΔR8.74 (for packaging) and pMD2.G (VSV-G, for envelope). In all cases, medium containing the viral particles was collected after 24 h and filtered (0.45 μm filters). For transduction, viral particles were diluted in Polybrene containing medium (8 ng·μl−1, Sigma Aldrich). Medium was refreshed 24 h after the transduction and selection of transduced cells was achieved through antibiotic selection (puromycin, 3 μg·ml−1, Sigma Aldrich). Stable pools were validated by WB analysis.

### Western Blotting

Whole cell extracts were prepared starting from lysed cultured cells (80% confluency). Protein concentration was determined using the Bio-Rad DC protein assay (Bio-Rad). These extracts were run on 4-12% Bis-Tris Bolt or NuPAGE precast gels in MES/MOPS running buffer (Invitrogen), followed by transfer on nitrocellulose membranes (Life Technologies). Of note, for APP-CTFs characterization, samples were run on 16% tricine gels (Life Technologies) in the appropriate running buffer (LC1675, Life Technologies). Membranes were blocked with 5% non-fat milk (1 h, RT), incubated with primary antibodies (overnight, 4° C.), rinsed and incubated with HRP-conjugated secondary antibodies (1 h, RT). Immuno-detection was carried out after rinsing using Western Lightning-Plus ECL reagent (NEL105001EA, PerkinElmer), and immunoreactive protein bands were digitally imaged on the Fuji MiniLAS 3000 imager (Fuji, Düsseldorf, Germany). Analysis was carried out using the Aida Image Analyzer software (Raytest, Germany) or Image J (Schindelin et al 2012; Schneider et al 2012).

EndoH-EndoF assay: The EndoH-EndoF assay was carried out as described in Coen et al 2012. Briefly, cell extracts were treated with either EndoH or EndoF (P0702L and P0705L, Bioke, New England Biolabs), according to the manufacturer's protocol. Cell extracts (40 μg) were loaded on 4-20% Tris Glycine gels (Novex, ThermoFisher Scientific) and transferred on PVDF membranes.

### Calcium Dynamics

Cytosolic response: Ca2+ responses were measured as previously described in Coen et al 2012. Briefly, cells (0.005 to 0.01×106) were seeded two to three days prior to the experiment on glass coverslips (631-0153, VWR). On the day of recording, cells were rinsed three times in Ringer solution (155 mM NaCl, 5 mM KCl, 2 mM MgCl2, 2 mM CaCl2, 10 mM Glucose, 10 mM HEPES, 2 mM NaH2PO4·2H20; pH 7.3), and loaded 1 μM Fura2-AM (F1221, ThermoFisher Scientific) for 30 min (RT). Cells were rinsed three times and left to equilibrate for at least 15-20 min (RT). All acquisitions were carried out in Ringer Ca2+ free solution (same as above, albeit the 2 mM CaCl2 were replaced by 2 mM EGTA). During experiments, cells were challenged with different drugs, such as Thapsigargin (T7459, ThermoFisher Scientific, 1 μM final), GPN (ab145914, abcam, 100 μM final for MEFs, 500 μM for neurons), MLSA1 (Cat. No. 4746, Tocris, 20 μM final) or lonomycin (10634, Sigma Aldrich, 1 μM final). Images were acquired every 5 sec for 8-10 min, using an Olympus IX81 using an UAPO/340 40× oil objective (1.35 NA), operated by CellR Software (Olympus). Recordings were done using 340 and 380 nm excitation, and 530 nm emission filters. Image processing was performed using Image J Software (Schindelin et al 2012; Schneider et al 2012). Briefly, cells were segmented manually and fluorescence intensities were measured over time. Fura-2 signals were corrected to obtain ΔF/F0 (F0 being the initial signal recorded). For each cell, the area under the curve, corresponding to the aforementioned stimulation, was measured using GraphPad Prism.

Lysosomes-ER refilling assay: The same approach as in Garrity et al 2016 was used. Briefly, cells loaded with Fura2-AM were repeatedly challenged with GPN (100 μM—for 5 min). These stimulations were done in absence of extracellular Ca2+ (free Ca2+ Ringer buffer), to ensure that the monitored Ca2+ response could be attributed to intracellular lysosomal Ca2+ (Garrity et al 2016; Yuan et al 2021). In between stimulation, Ca2+ containing Ringer was perfused (for 15 min) to allow the resealing and the refilling of the ruptured lysosomes (Garrity et al 2016; Kilpatrick et al 2013). Analysis were performed as described in the above section, and the ratio of the area under curved measured after second GPN stimulation over the area under curve measured after the first GPN stimulation were computed (ratio GPN2/GPN1).

### Lysosomal pH Measurement

Lysosomal pH measurement were performed as described in (Canton and Grinstein 2015). Briefly, one day prior to the experiment cells plated on coverslips (631-0153, VWR) were pulsed for 2 h with 0.2 mg·ml−1 of Fluorescein-dextran, 10 000 MW (Sigma-Adrich), extensively rinsed and incubated overnight to allow the fluorescein-dextran to label lysosomes. All acquisitions were carried out at 37° C. in HBSS medium supplement with Ca2+ and Mg2+ but without phenol red (ThermoFisher/Invitrogen), on an inverted microscope (IX81; Olympus) equipped with an 100×/1.35 NA oil objective lens and Full camera (Olympus soft imaging solution). The setup was operated by Cell\hat{ }R software (Olympus). Recordings were done with 440 and 490 nm excitation and 520 nm emission filters. A calibration curve was obtained by perfusing cells with a solution of known pH (ranging from 4.0 to 7.0). Cells were treated with ionophores (10 μM nigericin (N1495, ThermoFisher Scientific) and 10 μM monensin (M5273, Sigma Aldrich)). Analysis was carried out using Image J (Schindelin et al 2012; Schneider et al 2012): to measure the lysosomal pH ratio, the 490 nm signal (background corrected) was normalized by the 440 nm ratio (also background subtracted). All values were averaged to estimate the mean lysosomal pH values in a given cell.

### Isolation of Late Endosomes/Lysosomes Using Super Paramagnetic Iron Oxide Nanoparticles

LE/LYS were isolated according to (Tharkeshwar et al 2017) with some modifications. Briefly, confluent cells were incubated with DMSA-coated SPIONs suspended in culture medium (0.2 mg·ml−1) for 30 min at 37° C. Excess of DMSA-coated SPIONs was washed using PBS and cells were re-incubated overnight in a humidified chamber with 5% CO2 at 37 C. Acidic washes (0.15M glycine, pH 3) were performed to remove SPIONs at the cell surface, and were followed by PBS washes. Cells were then scraped, centrifuged (180 g, 10 min) and pellets were resuspended in homogenization buffer (HB; 250 mM sucrose, 5 mM Tris and 1 mM EGTA pH 7.4 supplemented with PI). After cell cracking (12 passages, clearance 10 μm) the total homogenate was centrifuged (800×g, 10 min) and the post nuclear supernatant (PNS) was loaded on a LS column (pre-equilibrated with HB) placed in a strong magnetic field (SuperMACSII, Miltenyi). Extensive washes with cold HB allowed to remove debris or organelles devoid of DMSA-SPIONs. Removing the magnetic field allowed to elute the bound fraction (containing LE/Lys) with HB. The collected fraction was then centrifuged (126000, 1 h) and the resulting pellet resuspended in 200 μl HB. To investigate the normal formation of the v-ATPase in the purified fractions the ratio of the V0/V1 domains was evaluated by WB (as in (Serra-Peinado et al 2016) and in (Lafourcade et al 2008)). In here, equal amounts of protein were denatured in sample buffer (Life Technologies) and loaded on gels.

### Suspension Adhesion Assay

Adherent cells were incubated on ice (15 min) with 10 μg·ml−1 CTxB-Alexa488 in PBS−/−. Cells were detached with trypsin, washed and held in suspension with 1% methylcellulose before re-plating on 20 μg·ml−1 fibronectin-coated coverslips. At defined time-points, internalization and endocytic transport/recycling of CTxB-Alexa488 was analyzed by immuno-cytochemistry. For the analysis, the number of cells displaying repolarized markers (Cav1) reported to the total cells was estimated per fields of view.

### Confocal Microscopy

Immunofluorescence: Cells were fixed (4% paraformaldehyde (PFA)/4% sucrose in PBS free of Ca2+ and Mg2+ (PBS−/−) 20 min, RT), permeabilized (0.1% triton X-100 in in PBS−/−; 10 min, RT), and blocked (2% BSA, 2% FBS, 1% gelatin, and depending of the experiment 2% of goat or donkey serum (Bioké or Sigma), 1 h RT). Primary antibodies diluted in the same blocking buffer were incubated overnight at 4° C. Following PBS washing steps, (goat- or donkey-) secondary antibodies conjugated with Alexa fluor dyes (Alexa 488, 568, or 647, all ThermoFisher Scientific) diluted in the blocking buffer were incubated for 1 h, RT. Coverslips were then mounted with Mowiol (Sigma). When cholesterol labelling was performed, fixed cells were treated with 200 μg·ml−1 filipin (F4767, Sigma) for 2 h at RT, prior to permeabilization.

Acquisition: Z stack images (spacing 2 μm), or a median plan were acquired on a Nikon 1AR connected to an inverse Nikon Ti-2000 equipped with an oil-immersion plan APO 60× objective lenses with 1.40 NA. Data were collected using Nikon Imaging Software. For the representation sake, images were displayed in false color.

Image analysis: All image analysis was done using Image J software (Schindelin et al 2012; Schneider et al 2012). To measure the area of the organelle of interest (early endosomes-EEA1; lysosomes-Lamp1; retromer-VPS35 . . . ), segmentation was realized using automatic thresholding. The following masks were used: Mean (EEA1), Ostu (Lamp1), Intermodes (VPS35). Overlap of signal of interests were quantified with Manders colocalisation index (Plugin Jacob, (Bolte and Cordelieres 2006)). When indicated, co-localization data was normalized to the reference group for multiple comparisons.

### Airyscan Imaging

Fixed cells: Images of fixed transfected cells (Lamp1-mCherry and Sec61-GFP or VAPB-mCherry and Stard3) were acquired using an inverted Zeiss LSM 880 microscope with Airyscan detector in superresolution mode. The system was equipped with a 63×1.4 NA Plan-Apochromat objective lens and operated using Zen Black (version 2.3, Carl Zeiss Microscopy GmbH). The following excitation lasers Argon 488, 514, He—Ne 543, 594 and 633 were used in combination with the following filter sets combinations BP 420-480+BP 495-550, BP 420-480+BP 495-620, BP 420-480+LP 605, BP 465-505+LP 525, BP 495-550+LP 570, and BP 570-620+LP 645. Cells with similar levels of transfection were selected. All Airyscan acquired images were processed using the default values. Obtained images were further processed using the plot profile function of Image J (Schindelin et al 2012; Schneider et al 2012).

Live-imaging of hypotonic treated cells: Transfected cells (with APP-YFP or C99-GFP and Sec61-mCherry, or treated with mitotracker Deep Red) were treated with hypotonic media (5% DMEM in water, pH˜7, pre-equilibrated at 37° C. and 5% CO2) for 10 min at 37° C. to allow the formation of the ERLarge intracellular vesicles, as in King et al 2020. Treated cells were stable for 15 min before detaching. Images of transfected cells were acquired using the fast Airyscan mode of the LSM880 microscope, equipped with the same objective, lasers and filters as above. Acquired and processed images were analysed using the Image J plot profile feature (vide supra).

### Live-Imaging SIM

Live cell imaging with cells transiently transfected with mCherry-LAMP1 and Sec61-GFP were imaged with a two camera lattice SIM Elyra7 microscope (Carl Zeiss, Jena, Germany) equipped with a Plan Apo 63×1.4 NA objective lens. Dual color frames were acquired every 250 ms for about 2 min. SIM reconstruction was done with ZEN black software (Carl Zeiss, Jena, Germany). For diffusion analysis of lysosomes, ROIs of the same size were made for the whole stack of the lysosomal channel. Tracking was done using Metamorph PALMtracer software with Auto thresholding and circularity feature on (https://www.iins.u-bordeaux.fr/team-sibarita-PALMTracer). Only tracks with more than 8 points where considered, MSD was calculated by linear fitting of the first four points. MSD of individual tracks were averaged per cell. For type of motility analysis, the alpha was calculated by the power of the MSD. Immobile tracks where defined as those with the diffusion coefficient below the microscope limit of resolution. This was calculated by the average of MSD (0) of the control cells, rendering a limit of 0.0035 μ2/s. From the mobile tracks the alpha was classified as follows: less than 0.1=confined, between 0.1 and 0.9=abnormal, between 0.9 and 1.1=Brownian, and more than 1.1=directed (Sibarita, 2014). The D Log 10 was calculated to analyze different populations.

Colocalization analysis: Analysis has been performed on Fiji (Schindelin et al 2012) using an ImageJ custom macro. For the lysosome and ER channels, a bleach correction using exponential fit (Miura 2020) is first performed and then normalized using the CLIJX (Haase et al., 2020) plugin method (CLIJx_normalize from 0 to 1). A moment automatic threshold (Tsai 1985) is then performed to create a binary mask. Objects smaller than 20 px are excluded and the area of the lysosome and ER is then measured by time point. A Boolean AND operation using the Image Calculator in Fiji is performed to create the overlapping image mask between lysosome and ER and the overlapping area is measured by time point. In addition, the mean through all the timepoints of the lysosome, ER and overlapping area are computed.

### Transmission Electron microscopy

Classical TEM: MEF cells treated as described previously (Tharkeshwar et al 2017). Briefly, cells were grown in 35 mm culture dishes till 90-95% confluency, fixed in 2.5% glutaraldehyde (Agar Scientific) (overnight, 4° C.) and rinsed with 0.1 M sodium cacodylate buffer (pH 7.2) to remove glutaraldehyde. Fixed cells were scrapped in the washing buffer, centrifuged (200 g, RT), resuspended in 1.5% agarose, centrifuged again (400 g, RT) before being left on ice to solidify (30 min). Samples were post-fixed in 1% osmium tetroxide and 1.5% ferrocyanide (2 h), rinsed with dH2O and dehydrated in a graded ethanol series (from 30-100%). Samples were en bloc stained with 4% uranyl acetate in the 70% ethanol step (30 min, 4° C.). Following dehydration, samples were infiltrated with epoxy resin/propylene oxide mixtures (50% and 66%). The next day, cell pellets were embedded with 100% epoxy resin in inverted BEEM-capsules (2 days, 60° C.). Ultrathin sections of 70 nm were cut using an ultratome (LEICA REICHERT Ultracut S) and post-stained with 4% uranyl acetate in water (10 min) and Reynolds' lead citrate (Reynolds 1963) (5 min). Micrographs were taken on a JEOLJEM 1400 electron microscope equipped with an Olympus Quemesa 11 Mpxl camera at 80 KV.

KDEL-HRP TEM: Cells were fixed in 1.3% glutaraldehyde (Agar Scientific) in 0.1M sodium cacodylate buffer (pH 7.2) for 1 h at room temperature. Cells were washed to remove glutaraldehyde using 0.1M sodium cacodylate buffer and incubated for in 0.1M ammoniumphosphate (pH 7.4) (10 min, RT). The DAB reaction mixture obtained by dissolving 3′ 3-diaminobenzidine tetrahydrochloride (DAB) in 0.1M ammoniumphosphate (0.5 mg/ml) was further filtered (0.2 μm) to remove any undissolved precipitate and used to wash the cells (10 min, RT). The initial ammoniumphosphate/DAB mixture was replaced with ammoniumphosphate/DAB mixture with 0.005% H2O2 to generate the insoluble reaction product (15 min, RT). Samples were post-fixed with 1% osmium tetroxide and 1.5% ferrocyanide (1h) and extensively washed with 0.1M sodium cacodylatebuffer and ddH2O before being en bloc stained with 0.5% uranyl acetate in ddH2O (overnight, 4° C.). Samples were dehydrated in a graded ethanol series (from 30-100%) and further infiltrated in epoxy resin/ethanol mixtures (50% and 66%). As for classical TEM, samples were embedded in epoxy resin (2 days, 60° C.); ultrathin sections of 70 nm were cut on an ultratome (LEICA REICHERT Ultracut S) and post-stained with 4% uranyl acetate (10 min) and Reynolds lead citrate (5 min). For quantification, the distance between KDEL-HRP ER membrane and LE/Lys were measured on EM images using the RADIUS 2.0 (Build 14402, Emsis) software.

### Statistics and Reproducibility

Experiments shown in figures usually correspond to pool of experiments (n=number of cells analyzed, N=number of experiments). All graphs and statistical analyses were performed using GraphPad Prism (versions 7 or higher), and exact p values are indicated in the figures. Data are usually represented as (i) boxes and whiskers; where bars include 90% of the points, the line represents the median, and the box contains 75% of the data or as (ii) mean±SEM. Comparisons between any two groups were done using the non-parametric Mann-Whitney statistical test, and comparison between multiple groups were realized using Multiple Anova Kruskal-Wallis with Dunn's post-test. Experiments were not randomized and the investigators were not blinded to allocation during experiments and outcome assessment.

Sequence Listing
SEQ ID NO: 1 depicts the amino acid sequence of the human Amyloid beta precursor
protein (APP).
MLPGLALLLLAAWTARALEVPTDGNAGLLAEPQIAMFCGRLNMHMNVQNGKWDSDPSGTKTCIDTKEGILQYCQE
VYPELQITNVVEANQPVTIQNWCKRGRKQCKTHPHFVIPYRCLVGEFVSDALLVPDKCKFLHQERMDVCETHLHWH
TVAKETCSEKSTNLHDYGMLLPCGIDKFRGVEFVCCPLAEESDNVDSADAEEDDSDVWWGGADTDYADGSEDKVVE
VAEEEEVAEVEEEEADDDEDDEDGDEVEEEAEEPYEEATERTTSIATTTTTTTESVEEVVRVPTTAASTPDAVDKYLETP
GDENEHAHFQKAKERLEAKHRERMSQVMREWEEAERQAKNLPKADKKAVIQHFQEKVESLEQEAANERQQLVETH
MARVEAMLNDRRRLALENYITALQAVPPRPRHVFNMLKKYVRAEQKDRQHTLKHFEHVRMVDPKKAAQIRSQVMT
HLRVIYERMNQSLSLLYNVPAVAEEIQDEVDELLQKEQNYSDDVLANMISEPRISYGNDALMPSLTETKTTVELLPVNG
EFSLDDLQPWHSFGADSVPANTENEVEPVDARPAADRGLTTRPGSGLTNIKTEEISEVKMDAEFRHDSGYEVHHQKL
VFFAEDVGSNKGAIIGLMVGGVVIATVIVITLVMLKKKQYTSIHHGVVEVDAAVTPEERHLSKMQQNGYENPTYKFFE
QMQN
SEQ ID NO: 2 depicts the amino acid sequence of the human Amyloid beta precursor
protein C99 (APP-C99).
DAEFRHDSGYEVHHQKLVFFAEDVGSNKGAIIGLMVGGVVIATVIVITLVMLKKKQYTSIHHGVVEVDAAVTPEERHL
SKMQQNGYENPTYKFFEQMQN
SEQ ID NO: 3 depicts the amino acid sequence of the human Amyloid beta precursor
protein C83 (APP-C83).
LVFFAEDVGSNKGAIIGLMVGGVVIATVIVITLVMLKKKQYTSIHHGVVEVDAAVTPEERHLSKMQQNGYENPTYKFF
EQMQN
SEQ ID NO: 4 depicts the amino acid sequence of the human Ab38 fragment
DAEFRHDSGYEVHHQKLVFFAEDVGSNKGAIIGLMVGG
SEQ ID NO: 5 depicts the amino acid sequence of the human Ab40 fragment
DAEFRHDSGYEVHHQKLVFFAEDVGSNKGAIIGLMVGGVV
SEQ ID NO: 6 depicts the amino acid sequence of the human Ab42 fragment
DAEFRHDSGYEVHHQKLVFFAEDVGSNKGAIIGLMVGGVVIA
SEQ ID NO: 7 depicts the amino acid sequence of the human Ab43 fragment
DAEFRHDSGYEVHHQKLVFFAEDVGSNKGAIIGLMVGGVVIAT
SEQ ID NO: 8 depicts the amino acid sequence of the NPC1 protein
MTARGLALGLLLLLLCPAQVFSQSCVWYGECGIAYGDKRYNCEYSGPPKPLPKDGYDLVQELCPGFFFGNVSLCCDVR
QLQTLKDNLQLPLQFLSRCPSCFYNLLNLFCELTCSPRQSQFLNVTATEDYVDPVTNQTKTNVKELQYYVGQSFANAM
YNACRDVEAPSSNDKALGLLCGKDADACNATNWIEYMFNKDNGQAPFTITPVFSDFPVHGMEPMNNATKGCDESV
DEVTAPCSCQDCSIVCGPKPQPPPPPAPWTILGLDAMYVIMWITYMAFLLVFFGAFFAVWCYRKRYFVSEYTPIDSNI
AFSVNASDKGEASCCDPVSAAFEGCLRRLFTRWGSFCVRNPGCVIFFSLVFITACSSGLVFVRVTTNPVDLWSAPSSQA
RLEKEYFDQHFGPFFRTEQLIIRAPLTDKHIYQPYPSGADVPFGPPLDIQILHQVLDLQIAIENITASYDNETVTLQDICLA
PPSPYNTNCTILSVLNYFQNSHSVLDHKKGDDFFVYADYHTHFLYCVRAPASLNDTSLLHDPCLGTFGGPVFPWLVLG
GYDDQNYNNATALVITFPVNNYYNDTEKLORAQAWEKEFINFVKNYKNPNLTISFTAERSIEDELNRESDSDVFTVVIS
YAIMFLYISLALGHMKSCRRLLVDSKVSLGIAGILIVLSSVACSLGVFSYIGLPLTLIVIEVIPFLVLAVGVDNIFILVQAYQRD
ERLQGETLDQQLGRVLGEVAPSMFLSSFSETVAFFLGALSVMPAVHTFSLFAGLAVFIDFLLQITCFVSLLGLDIKRQEK
NRLDIFCCVRGAEDGTSVQASESCLFRFFKNSYSPLLLKDWMRPIVIAIFVGVLSFSIAVLNKVDIGLDQSLSMPDDSYM
VDYFKSISQYLHAGPPVYFVLEEGHDYTSSKGQNMVCGGMGCNNDSLVQQIFNAAQLDNYTRIGFAPSSWIDDYFD
WVKPQSSCCRVDNITDQFCNASVVDPACVRCRPLTPEGKQRPQGGDFMRFLPMFLSDNPNPKCGKGGHAAYSSAV
NILLGHGTRVGATYFMTYHTVLQTSADFIDALKKARLIASNVTETMGINGSAYRVFPYSVFYVFYEQYLTIIDDTIFNLGV
SLGAIFLVTMVLLGCELWSAVIMCATIAMVLVNMFGVMWLWGISLNAVSLVNLVMSCGISVEFCSHITRAFTVSMK
GSRVERAEEALAHMGSSVFSGITLTKFGGIVVLAFAKSQIFQIFYFRMYLAMVLLGATHGLIFLPVLLSYIGPSVNKAKSC
ATEERYKGTERERLLNF
SEQ ID NO: 9 depicts the amino acid sequence of the NPC2 protein isoform1
MRFLAATFLLLALSTAAQAEPVQFKDCGSVDGVIKEVNVSPCPTQPCQLSKGQSYSVNVTFTSNIQSKSSKAVVHGIL
MGVPVPFPIPEPDGCKSGINCPIQKDKTYSYLNKLPVKSEYPSIKLVVEWQLQDDKNQSLFCWEIPVQIVSHL
SEQ ID NO: 10 depicts the amino acid sequence of the NPC2 protein isoform2
MRFLAATFLLLALSTAAQAEPVQFKDCGSVDGVIKEVNVSPCPTQPCQLSKGQSYSVNVTFTSNIQSKSSKAVVHGIL
MGVPVPFPIPEPDGCKSGINCPIQKDKTYSYLNKLPVKSEYPSVSHL

### CITATION LIST

Alpy et al 2013 J Cell Sci 126, 5500-5512.
Alves da Costa et al 2006 J Neurosci 26, 6377-6385.
Andersen et al 2005 Proc Natl Acad Sci U S A 102, 13461-13466.
Annaert et al 1999 The Journal of cell biology 147, 277-294.
Balasubramanian et al 2007 Nat Cell Biol 9, 1381-1391.
Bolte and Cordelieres 2006 J Microsc 224, 213-232.
Borg et al 1996 Mol Cell Biol 16, 6229-6241.
Bray 2016 Nat Rev Mol Cell Biol 17, 722-735.
Burgett et al 2011 Nat Chem Biol 7, 639-647.
Burgoyne et al 2015 Biochim Biophys Acta 1853, 2012-2017.
Canton and Grinstein 2015 Methods Cell Biol 126, 85-99.
Cao and Sudhof 2001 Science 293, 115-120.
Coen et al 2012 The Journal of cell biology 198, 23-35.
Currinn et al 2016 Cell Mol Life Sci 73, 393-408.
De Strooper and Annaert 2010 Annu Rev Cell Dev Biol 26.
De Strooper and Karran 2016 Cell 164, 603-615.
Di Mattia et al 2020 EMBO J 39, e104369.
Escamilla-Ayala et al 2020 Elife 9.
Esselens et al 2004 The Journal of cell biology 166, 1041-1054.
Garrity et al 2016 Elife 5.
Giordano et al 2013 Cell 153, 1494-1509.
Haass and Selkoe 2007 Nat Rev Mol Cell Biol 8.
Hardy and Allsop 1991 Trends Pharmacol Sci 12
Hoque et al 2020 Int J Mol Sci 21.
Howell et al 1999 Mol Cell Biol 19, 5179-5188.
Huan et al 2020 Front Cell Dev Biol 27.
Hung and Livesey 2018 Cell Rep 25, 3647-3660 e3642.
Huotari and Helenius 2011 EMBO J 30, 3481-3500.
Jadot et al 1984 Biochem J 219, 965-970.
Jurisch-Yaksi et al 2013 Biochim Biophys Acta 1828, 2815-2827.
Kessissoglou et al 2020 PLOS Biol 18, e3000703.
Kilpatrick et al 2013 J Cell Sci 126, 60-66.
Kim et al 2016 Mol Psychiatry 21, 707-716.
Kimberly et al 2001 J Biol Chem 276, 40288-40292.
King et al 2020 Proc Natl Acad Sci U S A 117, 7225-7235.
Kopan and Ilagan 2004 Nat Rev Mol Cell Biol 5, 499-504.
Kwart et al 2019 Neuron 104, 256-270 e255.
Lafourcade et al 2008 PLOS One 3, e2758.
Lee et al 2015 Cell Rep 12, 1430-1444.
Ma and Mayr 2018 Cell 175, 1492-1506 e1419.
Marambaud et al 2003 Cell 114, 635-645.
Miura 2020 F1000Res 9, 1494.
Montesinos et al 2020 EMBO J, e103791.
Morgan et al 2020 Cell Calcium 86, 102139.
Muller et al 2007 Mol Biol Cell 18, 201-210.
Netzer et al 2003 Proc Natl Acad Sci U S A 100, 12444-12449.
Nyabi et al 2003 J Biol Chem 278, 43430-43436.
Pardossi-Piquard et al 2005 Neuron 46, 541-554.
Patterson et al 2007 Lancet Neurol 6
Pera et al 2017 EMBO J 36, 3356-3371.
Pineda et al 2019 Orphanet J Rare Dis 14
Raemaekers et al 2012 EMBO J 31, 3252-3269.
Reynolds 1963 The Journal of cell biology 17, 208-212.
Sardi et al 2006 Cell 127, 185-197.
Schikorski et al 2007 J Neurosci Methods 165, 210-215.
Schindelin e al 2012 Nat Methods 9, 676-682.
Schneider et al 2012 Nat Methods 9, 671-675.
Selkoe and Hardy 2016 EMBO Mol Med 8, 595-608.
Serra-Peinado et al 2016 J Biol Chem 291, 7286-7299.
Shen et al 2012 Nat Commun 3, 731.
Shibata et al 2008 J Biol Chem 283, 18892-18904.
Shioi et al 2007 J Neurochem 101, 674-681.
Singhal et al 2020 Sci Rep 10, 8663.
Sitarska et al 2021 Metabolic Brain Disease 272
Spasic et al 2006 J Biol Chem 281, 26569-26577.
Spasic et al 2007 The Journal of cell biology 176, 629-640.
Stamer et al 2002 The Journal of cell biology 156, 1051-1063.
Szaruga et al 2017 Cell 170 e414.
Tharkeshwar et al 2017 Sci Rep 7, 41408.
Thiele and Lipsky 1990 PNAS 87.
Tsai 1985 Computer Vision, Graphics, and Image Processing 29, 377-393.
Vite et al 2015 Sci Transl Med 7
Wilhelm et al 2019 Intracellular and Plasma Membrane Cholesterol Labeling and Quantification Using Filipin and GFP-D4. In: Drin G. (eds) Intracellular Lipid Transport. Methods in Molecular Biology, vol 1949. Humana Press, New York, NY.
Wraith et al 2010 Mol Genet Metab 99
Xu et al 2016 J Clin Invest 126, 1815-1833.
Yang et al 2019 Protein Cell 10, 8-19.
Yuan et al 2021 J Cell Sci 134.
Zervas et al 2001 Curr Biol 11
Zewe et al 2018 Elife 7.

1. A method of detecting an improved amyloid precursor protein (APP) processing modulator from a collection of gamma-secretase modulators, the method comprising:
administering a gamma-secretase modulator from the collection to one or more cells expressing a gamma-secretase complex and comprising a polypeptide having at least 95% amino acid identity to SEQ ID No. 1;
quantifying the level of APP-carboxyterminal fragment (CTF) in the one or more cells before and after administering the gamma-secretase modulator;
optionally determining the Ca2+ level and/or the cholesterol level in the lysosomes of the one or more cells before and after administering the gamma-secretase modulator;
detecting that the APP-CTF level is at most 50% increased after the administration of the gamma-secretase modulator compared to before the administration and optionally if the Ca2+ level in the lysosomes is at most 20% decreased and/or the cholesterol level in the lysosomes is at most 25% increased after the administration of the gamma-secretase modulator compared to before the administration.

2. A method of detecting an improved APP processing modulator, the method comprising:
providing one or more cells expressing a functional gamma-secretase complex and comprising a polypeptide having at least 95% amino acid identity to SEQ ID No. 1 hereof;
quantifying amyloid beta (Ab) peptides with a length of 38, 40, 42 and/or 43 amino acids produced in said one or more cells before and after administering a test compound;
quantifying before and after administering the test compound the level of APP-CTF in the one or more cells and/or the Ca2+ level in the lysosomes of the one or more cells and/or the cholesterol level in the lysosomes of the one or more cells;
detecting a statistically significant increase in the ratio of Ab38/Ab42, Ab40/Ab42, Ab40/Ab43 or of Ab (38+40)/Ab (42+43) compared to before administering the test compound and at most a 50% increased APP-CTF level and/or at most a 20% decrease in lysosomal Ca2+ level and/or at most a 25% increase lysosomal cholesterol level after the administration of the test compound compared to before administration.

3. The method according to claim 1 wherein the APP-CTF level is quantified via immune-based assays.

4. The method according to claim 1, wherein the APP-CTF is fluorescently labelled and wherein the APP-CTF level corresponds to the APP-CTF fluorescent signal.

5. The method according to claim 2, wherein Ab peptides are quantified via immune-based assays.

6. The method according to claim 1, wherein the Ca2+ and/or cholesterol level in the lysosomes is determined by an image-based assay.

7. The method according to claim 1, wherein the lysosomes are visualised by LAMP1.

8. The method according to claim 1, wherein the one or more cells are mammalian cells.

9. (canceled)

10. (canceled)

11. The method according to claim 1 wherein the APP-CTF level is quantified via immune-based assays.

12. The method according to claim 2, wherein the APP-CTF is fluorescently labelled and wherein the APP-CTF level corresponds to the APP-CTF fluorescent signal.

13. The method according to claim 2, wherein the Ca2+ and/or cholesterol level in the lysosomes is determined by an image-based assay.

14. The method according to claim 2, wherein the lysosomes are visualised by LAMP1.

15. The method according to claim 2, wherein the one or more cells are mammalian cells.

16. The method according to claim 1 wherein the APP-CTF level is quantified via immune-based assays.


SEQUENCE LISTING (18 sequences)

SEQ ID NO: 1 - protein, 695 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 2 - protein, 99 aa, Homo sapiens
DAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 3 - protein, 83 aa, Homo sapiens
LVFFAEDVGSNKGAIIGLMVGGVVIATVIVITLVMLKKKQYTSIHHGVVEVDAAVTPEERHLSKMQQNGYENPTYKFFEQMQN

SEQ ID NO: 4 - protein, 38 aa, Homo sapiens
DAEFRHDSGYEVHHQKLVFFAEDVGSNKGAIIGLMVGG

SEQ ID NO: 5 - protein, 40 aa, Homo sapiens
DAEFRHDSGYEVHHQKLVFFAEDVGSNKGAIIGLMVGGVV

SEQ ID NO: 6 - protein, 42 aa, Homo sapiens [sequence omitted]

SEQ ID NO: 7 - protein, 43 aa, Homo sapiens
DAEFRHDSGYEVHHQKLVFFAEDVGSNKGAIIGLMVGGVVIAT

SEQ ID NO: 8 - protein, 1278 aa, Homo sapiens [sequence omitted]

SEQ ID NO: 9 - protein, 151 aa, Homo sapiens
MRFLAATFLLLALSTAAQAEPVQFKDCGSVDGVIKEVNVSPCPTQPCQLSKGQSYSVNVTFTSNIQSKSSKAVVHGILMGVPVPFPIPEPDGCKSGINCPIQKDKTYSYLNKLPVKSEYPSIKLVVEWQLQDDKNQSLFCWEIPVQIVSHL

SEQ ID NO: 10 - protein, 125 aa, Homo sapiens
MRFLAATFLLLALSTAAQAEPVQFKDCGSVDGVIKEVNVSPCPTQPCQLSKGQSYSVNVTFTSNIQSKSSKAVVHGILMGVPVPFPIPEPDGCKSGINCPIQKDKTYSYLNKLPVKSEYPSVSHL

SEQ ID NO: 11 - DNA, 25 bp, Mus musculus
  source 1..25: mol_type = genomic DNA
caacgttatcaagtacctccccgaa

SEQ ID NO: 12 - DNA, 25 bp, Mus musculus
  source 1..25: mol_type = genomic DNA
caacgtcctgggcgaccgtcgggcc

SEQ ID NO: 13 - DNA, 23 bp, synthetic construct
  source 1..23: mol_type = other DNA
gtacccactgatggcaacgccgg

SEQ ID NO: 14 - DNA, 23 bp, synthetic construct
  source 1..23: mol_type = other DNA
acggtaaggaatcacgatgtggg

SEQ ID NO: 15 - protein, 50 aa, synthetic construct
VMLKKKQYTSIHHGVVEVDAAVTPEERHLSKMQQNGYENPTYKFFEQMQN

SEQ ID NO: 16 - protein, 66 aa, synthetic construct
MGCIKSKRKDNLNDDGVDNKKKQYTSIHHGVVEVDAAVTPEERHLSKMQQNGYENPTYKFFEQMQN

SEQ ID NO: 17 - protein, 66 aa, synthetic construct
MGCIKSKRKDNLNDDGVDNKKKQYTSIHHGVVEVDAAVTPEERHLSKMQQNGAENPTAKFFEQMQN

SEQ ID NO: 18 - protein, 66 aa, synthetic construct
MGCIKSKRKDNLNDDGVDNMVSKGEELFTGVVPILVELDGDVNGHRFSVSGEGEGDATYGKLTLKF